(19) United States
(12) Patent Application Publication
(10) Pub. No.: US 2023/0056380 A1
(43) Pub. Date: Feb. 23, 2023

(54) METHODS AND KIT FOR DETECTION OF ANALYTES

(71) Applicant: Randy Leiman Allen, Apex, NC (US)

(72) Inventor: Randy Leiman Allen, Apex, NC (US)

(21) Appl. No.: 17/795,264

(22) PCT Filed: Mar. 31, 2021
(86) PCT No.: PCT/US2021/025279

§ 371 Date: Jul. 26, 2022

Related U.S. Application Data
(60) Provisional application No. 63/135,699, filed on Jan. 10, 2021.
(60) Provisional application No. 63/122,307, filed on Dec. 7, 2020.
(60) Provisional application No. 63/040,340, filed on Jun. 17, 2020.
(60) Provisional application No. 62/968,648, filed on Jan. 31, 2020.

(51) Int. Cl. A61K 49/00

(52) U.S. Cl. CPC A61K 49/0006; A61B 5/411

(57) ABSTRACT

The present invention provides for a kit and methods that detect certain analytes of interest potentially present in blood and bodily fluids of a living mammal. The methods and kit encompass a bioassay performed in vivo. Contact of the bioassay reagent with the analyte, if present, renders a response that can be clinically assessed visually or by reading instrumentation or by biosensor. In one embodiment, the invention may be used to detect the presence, absence, or amount of suspected analyte present in a patient test subject. The invention is particularly suited for point-of-care (POC) use, self-testing, large-scale implementation and for use with patients where limited sample volumes are available or accessible.

### CROSS-REFERENCE TO RELATED APPLICATIONS

This application claims the benefit of U.S. Provisional Pat. Application Serial No. 62/968,648 filed on Jan. 31, 2020; U.S. Provisional Pat. Application Serial No. 63/040,340 filed on Jun. 17, 2020; U.S. Provisional Pat. Application Serial No. 63/122,307 filed on Dec. 7, 2020; U.S. Provisional Pat. Application Serial No. 63/135,699 filed on Jan. 10, 2021; and International Patent Appliction No. PCT/US2021/025279, filed Mar. 31, 2021, all of which are incorporated herein in their entirety by reference thereto.

### FIELD OF THE INVENTION

The present invention relates to methods, kits and reagents for determining the presence, absence, or amount of antibodies, receptors, receptor binding molecules, antigens, drugs, toxicants, toxins, pathogens, biomarkers, biochemicals, cell surface markers, and other molecules of interest present in blood and body fluids without the need for sample collection.

### BACKGROUND OF THE INVENTION

The global rise in infectious and noninfectious diseases and disease states coupled with a growing global population, has created a significant demand for new and innovative diagnostics. Conventional technologies, such as tests performed in centralized laboratories limit patient access, result in long sample turnaround times and contributes to “loss to follow up” and may not be sufficient for addressing the wellness of entire populations (Anuj Pathak, BCC Research Report HLC207A (2018)). While “point-of-care” (POC) tests overcome some of these limitations, both conventional and POC assays extensively utilize blood to determine the presence of diseases such as HIV/AIDS, malaria, dengue, cancer, cardiovascular, viral and bacterial infections. Notably, blood tests typically require sample collection, sample processing, sample stabilization, and frequently, sample transport. Additionally, pre-analytical sample preparation may add significant cost to lab-based and POC tests. Moreover, there is no guarantee that interferences potentially present in a sample have been removed or that samples have been properly handled, either of which may lead to false results.

Modern diagnostic tests are limited by the need for a sample that is collected and analyzed outside of living organisms. Pre-analytical errors account for up to 70% of all mistakes made in laboratory diagnostics, most of which arise from problems in sample preparation, collection, transportation, and preparation for analysis and storage (Plebani, M, Clin biochem Rev 33:85-87 (2012)). Collecting samples through vena puncture can be distressing to some patients, provides an opportunity for sample contamination, and creates a biohazard to healthcare workers. Collecting sufficient blood by fingerstick can be problematic, requiring the finger to be heated and/or massaged which in turn, can lead to problems with sample integrity. The limited amount of sample that can be collected from elderly, pediatric or frail patients may also impede or prevent the utilization of many blood-based diagnostic tests. Samples may also be lost or mislabeled. Accordingly, eliminating sample collection would be advantageous for blood-based assays.

By virtue of their nature, samples have a fixed volume with a fixed concentration of analyte available for testing thereby restricting the applicability of certain assays due to limits on their sensitivity thresholds. For example, “rapid diagnostic tests” (RDTs) utilizing finger prick blood provides an avenue to generate test results anywhere at any time, but the small volumes of blood used in these assays means less analyte available for detection, thereby limiting sensitivity. RDT devices and other POC technologies are employed throughout the world and have various advantages over conventional lab testing; however, limitations on accuracy and reliability of these tests exists due to limited sample size. Accordingly, it would be useful to have assay methods where sample size is not a constraint on assay performance.

While the burden of many diseases such as HIV/AIDS, tuberculosis, and hepatitis are heterogeneous, they disproportionately affect poorer demographics and “low and middle-income countries” (LMICs) worldwide (for review see Yuen et al., Nature Reviews, Disease Primers DOI:10.1038/nrdp.2018.35 (2018); Manns et al., “Hepatitis C Virus Infection” Nature Reviews, Disease Primers DOI:10.1038/nrdp.2017.6 (2017); Freiman, J.M., Ann. Intern. Med. 165(5):345 (2016); Pai et al., “Tuberculosis” Nature Reviews, Disease Primers DOI: 10.1038/nrdp.2016.76 (2016); Sewald et al., Curr. Opin. Cell Biol. 41:81 (2016); Park et al., Journal of Clinical Microbiology 48:2253 (2010)). The diagnostic landscape for these diseases and disease states has historically been dominated by complex, laboratory-based technologies, which are unable to meet the entire testing need in resource-limited settings. Complex technologies typically require significant capital investment, sophisticated laboratory infrastructure and highly trained technicians that are not available in many settings often resulting in limited geographic reach. As a result, most patients in LMICs do not have access to or the benefit of this type of testing where they receive care. Sadly, the fortunate few who may have access to complex testing seldom receive timely results from conventional laboratories, often taking days, weeks, or even months. Such delays frequently lead to poor patient outcomes or even death. Importantly, a patient’s unawareness of his or her own health status is considered to be a significant factor in the propagation and transmission of infectious diseases (Drain et al., Clinical Microbiology 32(3):1-25 (2019)).

While RDTs have transformed the testing landscape, even the simplest RDT has multiple steps often with multiple reagents being added and washed away or separated at different points in the assay. This consequently makes them difficult to use in the field. Accordingly, efforts to test hard to reach populations has been difficult to implement. Moreover, widespread use of self-testing is limited because patients are required to collect his or her own blood sample or to perform a mouth swab to obtain results. As such, it would be useful to have simple to use tests that are easily interpreted to provide the patient and healthcare workers with real-time, on-site results without requiring the patient to collect their own sample for testing (Mugambi et al., BMJ Glob Health DOI:10.1136/bmjgh-2018-000914 (2018); Drain et al, Lancet 14(3):239 (2014)).

In addition to the foregoing, there are major constraints encountered when designing mechanism of action studies such as limitations on the volume of biological specimens available for study. Robust and validated assays that can reliably and reproducibly assess immune function, disease states or effects of therapy with limited samples are not available. Further, the restricted amounts of tissue, cells and fluids that can be collected from elderly, pediatric or immunocompromised patients are often inadequate for the application of conventional assays to interrogate immune function.

Sample sparing assays that are needed but not available include assays for monitoring or assessment of antigen-specific immune responses, identifying the presence of distinct immune cell populations, measuring markers of T-cell turnover, detecting gene expression and assays for measuring mucosal inflammation and other innate immune responses (Department of Health and Human Services, SBIR, Phase 1 Program Solicitation PHS 2018-, NHI/NIAID 057)). Novel, sample sparing assays are needed to obtain maximal information from limited biological materials.

In addition to the various sample related problems such as inadequate sample size and sample collection issues, problems associated with delayed diagnosis due to the absence of, or the prompt delivery of, test results also present significant obstacles which are especially problematic in large populations potentially exposed to highly contagious and sometimes deadly pathogens. For example, diseases such as Eboli and Zika virus require a quick response time for diagnosing and managing outbreaks before they spiral out of control. Unfortunately, diagnosis of diseases such as Ebola shortly after infection can be difficult. Early symptoms of Eboli such as fever, headache, and weakness are not specific to Ebola virus infection and often are seen in patients with other more common, far less deadly diseases, like malaria and typhoid fever. Given the number of recent disease outbreaks like Ebola and Listeriosis, there is a dire need to strengthen health systems and ensure that public health threats are rapidly identified and contained. A rapid response test that can be quickly brought to market in large quantities for existing and future epidemics would clearly provide health care providers with significant advantages in time sensitive management of such outbreaks.

Additional problems arise when testing for diseases such as viruses, especially those hallmarked by latent seroconversion such as HIV. Millions of people are screened for HIV, HCV, and TB and test negative only to seroconvert after being tested. Moreover, healthcare workers active in the midst of a deadly disease outbreak, first responders reacting to a terrorist attack, or military personnel on the front lines are endangered by harmful agents even before presenting symptoms. Missed diagnoses and medical emergencies described above can be unpredictable and counter measures must incorporate development of new methods for the rapid detection, accurate diagnosis, and speedy treatment of exposed populations. For example, anthrax (Bacillus anthracis) is a highly toxic agent and can form spores that are extremely hearty and that can remain viable for a very long time. Once anthrax spores have been inhaled, the disease may progress rapidly often leading to death in a matter of days without timely treatment. Thus, early detection and differential diagnosis is critical. These groups and others would benefit from a sentinel tests that could be applied to a healthcare worker before exposure and detect subsequent exposure days, weeks, or even months later should they come in contact with that harmful agent. Such a test could save lives by detecting exposure at the earliest possible moment, containing outbreaks and stopping the spread of the disease.

Even diseases and pathogens that have been around for centuries are still prevalent and problematic, endangering entire populations. Accordingly, rapid and robust field tests would be beneficial to combat these health dilemmas as well. For example, according to the WHO in 2014, an estimated 9.6 million people developed active TB disease, 1.5 million of whom died. Between 5% and 15% of individuals infected with M. tuberculosis will progress (over months to a few years) to active TB disease, whereas the remainder with latent TB retain a persistent risk of developing active TB disease throughout their lifetime. In many settings, up to 50% of all people with culture-positive active TB disease do not have a prolonged productive (phlegm or mucus-producing) cough, and at least 25% have no symptoms whatsoever (Darin et al., Clinical Microbiology Reviews 31(4) DOI.org/10.1128/CMR.00021-18 (2018)).

The choice of a low-cost screening tool for TB is limited to detecting Latent TB Infections (LTBI). Two tests are available for the identification of LTBI: the TST and the Interferon-Gamma Release Assay (IGRA). The IGRA can also distinguish between BCG-induced and M. tuberculosis infection-induced positive TST responses (for review see Pai et al., “Tuberculosis” Nature Reviews, Disease Primers DOI:10.1038/nrdp.2016.76 (2016)).

The TST, performed using the Mantoux technique, consists of an intradermal injection of 2-5 tuberculin units (5 TU) of purified protein derivative (PPD). In a person who has cell-mediated immunity to these antigens, a delayed-type hypersensitivity reaction will occur within 48-72 hours. Interpretation of the TST takes into account the size of induration, the pre-test probability of M. tuberculosis infection and the risk of developing active TB disease if the person was truly infected.

Although the TST has several advantages, particularly in low-resource settings, including low reagent and equipment costs and limited skill and laboratory requirements, it has two major limitations. First, its specificity is compromised by late (that is, post-infancy) or repeated BCG vaccination (booster vaccinations) and, to a limited extent, by exposure to non-tuberculous mycobacteria. Second, it has limited predictive value. Most individuals with positive TST results do not progress to active TB disease.

In the early 2000s, IGRAs were introduced, with the hope to replace TSTs. IGRAs are in vitro blood tests of cell-mediated immune response: they measure T cell release of IFNγ following stimulation by RD1-encoded antigens (namely, the 6 kDa early secretory antigenic target and culture filtrate protein). RD1 antigens are more specific for M. tuberculosis than PPD antigens because they are not encoded in the genome of any BCG vaccine strains or of most species of non-tuberculous mycobacteria (exceptions are M. marinum, M. kansasii, Mycobacterium szulgai and Mycobacterium flavescens). However, like TSTs, IGRAs have poor predictive value.

After hundreds of research studies, it is clear that both the TST and the IGRA are acceptable but imperfect tests for LTBI. Neither test is able to accurately differentiate between LTBI and active TB disease nor to distinguish between new infections and re-infection events, a distinction that could be relevant in settings in which individuals who had previously received preventive therapy are at risk of becoming re-infected. In summary, none of the currently available LTBI tests meets the need for a highly predictive test that can help to identify the individuals who are at increased risk for the development of active TB disease and would, therefore, benefit most from LTBI therapy (preventive therapy). Notably, because all LTBI tests have low predictive value, widespread screening of low risk populations is counterproductive.

The detection of lipoarabinomannan (LAM) antigen in urine has emerged as a potential point-of-care test to detect HIV-associated active TB disease, with a modest reduction in mortality in only a highly selected group of hospitalized HIV-positive patients (see Sakamuri et al., Tuberculosis 93(3) DOI:10.1016/j.tube.2013.02.015 (2013); Abd el-Atty et al., Menoufia Medical Journal DOI: 10.4103/1110-2098.149720 (2014); Choudhary et al., Journal of Immunology DOI:10.4049/jimmunol.1701673 (2018); Youssef et al.,” Egyptian Journal of Bronchology DOI: 10.4103/1687-8426.193639 (2016)). A LAM rapid test for urine is now recommended by the WHO to assist and expedite the diagnosis of active TB disease in two specific populations: in HIV-positive adult in-patients with signs and symptoms of pulmonary and/or extrapulmonary TB who have a CD4+ T cell count of ≤100 cells per µ1, or HIV-positive patients who are seriously ill regardless of their CD4+ T cell count or with an unknown CD4+ T cell count.

Given the limitations of the available diagnostics screening tests for large populations for the detection of active TB, the development of new and better diagnostic tools for detection of active TB infections remains a priority. Several diagnostic tools are currently in development. Although these tools may seem robust and suitable for field testing conditions when taken at first glance, commercial versions of these products are typically designed for laboratory settings, making use of the only proven TB biomarker: bacterial nucleic acid sequences. Importantly, tests such as these often fall short regarding affordability and ease-of-use requirements when considering integration into primary care.

Recently, the World Health Organization (WHO) defined high-priority target product profiles (TPP) for TB diagnostics. These include a rapid non-sputum based test for detecting TB with the purpose of starting specific TB therapy on the same day. To be truly effective, tests such as these need to be performed in endemic settings with limited laboratory facilities, at a relatively low cost, using easily accessible samples that do not require the collection of body fluids and the disadvantages associated therewith. Accordingly, there is an urgent need to identify and capitalize on biomarkers (BM) that could be used in such tests and to develop novel testing formats that lead to improved and fast clinical decision making, one example being the development of improved tests that more accurately and differentially diagnose active TB disease (Goletti et al., Respirology 23:455 (2018); Correia-Neves et al., ERJ Open Res. DOI.org/10.1183/23120541.00115-2018 (2018)). There are no current universal methods for adapting newly found biomarkers to easily deployable devices.

Immunity consists of both a humoral (antibodies) and cell-mediated immunity which involves immune cells that specifically recognize, target and clear infected host cells. The immune status of a patient and the efficacy of vaccines rely on both humoral and cell-mediated immunity. Efforts to evaluate the immune response has typically focused on humoral immunity (the serum antibody response) because of the relative ease in which an antibody response can be measured by ELISA, neutralization assays, hemagglutination assays, etc. Because of the substantial role cell-mediated immunity plays in defense against infections, cancer, autoimmunity and a host of other diseases, new assay methods are needed. Current methods for analysis of cell-mediated immunity are complex and normally performed in a research setting in a biocontainment facility.

T cell responses are exquisitely antigen-specific, and they are at least as important as antibodies in defending vertebrates against infection. Indeed, most adaptive immune responses, including antibody responses, require helper T cells for their initiation. Most importantly, unlike B cells, T cells can help eliminate pathogens that reside inside host cells.

T cell responses differ from B cell responses in at least two crucial ways. First, T cells are activated by foreign antigen to proliferate and differentiate into effector cells only when the antigen is displayed on the surface of antigen-presenting cells in peripheral lymphoid organs. The T cells respond in this manner because the form of antigen they recognize is different from that recognized by B cells. Whereas B cells recognize intact antigen, T cells recognize fragments of protein antigens that have been partly degraded inside the antigen-presenting cell. The peptide fragments are then carried to the surface of the presenting cell on special molecules called the major histocompatibility complex (MHC) proteins, which present the fragments to T cells (Rock, K.L. et al., Trends Immunol. November 2016; 37(11): 724-737. Doi:10.1016/j.it.2016.08.010).

The second difference is that, once activated, effector T cells act only at short range, either within a secondary lymphoid organ or after they have migrated into a site of infection. They interact directly with another cell in the body, which they either kill or signal in some way (we shall refer to such cells as target cells). Activated B cells, by contrast, secrete antibodies that can act far away.

There are two main classes of T cells-cytotoxic T cells and helper T cells. Effector cytotoxic T cells directly kill cells that are infected with a virus or some other intracellular pathogen. Effector helper T cells, by contrast, help stimulate the responses of other cells-mainly macrophages, B cells, and cytotoxic T cells.

Superantigens are a distinct class of antigens that stimulate a primary T-cell response (Cheng, M.H. et al., PNAS, Oct. 13, 2020, vol 117, No 41, 25254-25262). Superantigens bind to both MHC and T-cell receptor molecules without intracellular processing that enables them to stimulate very large numbers of T cells. Superantigens are produced by many different pathogens, including bacteria, mycoplasmas, and viruses, and the responses they provoke are helpful to the pathogen rather than the host.

Superantigens are unlike other protein antigens, in that they are recognized by T cells without being processed into peptides that are captured by MHC molecules. Indeed, fragmentation of a superantigen destroys its biological activity, which depends on binding as an intact protein to the outside surface of an MHC class II molecule which has already bound peptide. In addition to binding MHC class II molecules, superantigens are able to bind the Vβ region of many T-cell receptors. Bacterial superantigens bind mainly to the Vβ CDR2 loop, and to a smaller extent to the Vβ CDR1 loop and an additional loop called the hypervariable 4 or HV4 loop. The HV4 loop is the predominant binding site for viral superantigens. Thus, the α-chain V region and the CDR3 of the β chain of the T-cell receptor have little effect on superantigen recognition, which is determined largely by the germline-encoded V sequences of the expressed β chain. Each superantigen is specific for one or a few of the different Vβ gene segments, of which there are 20-50 in mice and humans; a superantigen can thus stimulate 2-20% of all T cells.

This mode of stimulation does not prime an adaptive immune response specific for the pathogen. Instead, it causes a massive production of cytokines by CD4 T cells, the predominant responding population of T cells. These cytokines have two effects on the host: systemic toxicity and suppression of the adaptive immune response. Both these effects contribute to microbial pathogenicity. Among the bacterial superantigens are the staphylococcal enterotoxins (SEs), which cause food poisoning, and the toxic shock syndrome toxin-1 (TSST-1), the etiologic principle in toxic shock syndrome.

Easily assessable tests to detect and/or measure cell-mediated immunity and pathways would be of great benefit in the diagnosis and treatment of a disease and in vaccine production. There are no simple and effective diagnostics to measure cell-meidated immunity and associated pathologies.

Diagnosis and management of disease and disease states, be they related to genetics or exposure to pathological entities, is increasingly being facilitated by detection and measurement of biomarkers present in blood, urine, saliva, and other body fluids. Continuing progress in biomarker discovery has enabled early diagnosis, real-time monitoring, and improved disease management. However, urine and saliva are constrained by their limited number and variable concentration of biomarkers. Interstitial fluid (ISF) is another source of valuable and unique biomarkers but is difficult to sample from the body. Interstitial fluid surrounds cells and tissues throughout the body and is formed by extravasation of plasma from capillaries and modified by metabolic and other processes in the tissue. Nutrients and waste products are shuttled between blood vessels and cells by ISF which is roughly a combination of serum and cellular materials (see Kunder et al., Blood 118:5383 (2011); Hanen et al., International Immunology 27:219 (2015); Sewald et al., Curr. Opin Cell Biol. 41:81 (2016); Samant et al., Proc Natl Acad Sci USA 115:4583 (2018)).

Previous studies showed that 83% of proteins found in serum are also in ISF, but 50% of proteins in ISF are not in serum, suggesting that ISF may be a source of unique biomarkers as well as biomarkers found in blood. Many viruses are spread through the extracellular fluid consisting of interstitial fluid, lymph, and blood. Moreover, ISF may be suitable for continuous monitoring due to absence of clotting factors, as shown by commercial indwelling sensors for glucose that access ISF in the subcutaneous space. ISF is a better indicator of local tissue events as shown in tumor ISF collected from tissue biopsies. Skin is the most accessible organ and therefore an attractive source of ISF containing systemic and dermatological biomarkers.

ISF can currently be collected from skin using suction blisters by applying suction to skin at elevated temperature for up to 1 hour to create blisters filled with ISF. Dermal ISF can also be sampled by implantation of tubing in skin to collect ISF biomarkers by micro-dialysis or open-flow micro-perfusion, which require local anesthesia and expert training. The unique properties of ISF provides fertile grounds for assay development, but limited access to ISF prevents its use in assays. New tools and diagnostics tests are needed to exploit the benefits of detecting analytes in this medium.

Importantly, it will be further noted from the foregoing examples that most diseases and disease states as well as certain environmental and conflict based exposures are typically hallmarked by the presence of some associated analyte that is either directly or indirectly produced or present pursuant to or resulting from a pathological entity or genetic flaw. Such analytes present a tremendous opportunity for detection, diagnosis, treatment and prevention provided that they can be detected in a timely, relatively inexpensive manner. Alternatively, there are other situations where it would be advantageous to determine the absence, presence or amount of a substance such as hormones, growth factors, metabolites, etc. in order to confirm or establish baselines for the fitness, well-being or health state of a subject. Yet further still, it would be advantageous for health care workers, public health organizations, military personnel and others to have access to sentinel tests that could be applied before exposure and detect subsequent exposure days, weeks, or even months later should they come in contact with a harmful agent.

### SUMMARY OF THE INVENTION

The present invention provides for certain novel methods, reagent “constructs” and kits that can be readily employed by those skilled in the art to determine the presence, absence or amount of a broad spectrum of analytes, including but not limited to chemicals, peptides, proteins, lipids, carbohydrates, glycoproteins, nucleic acid sequences, or a combination thereof, the foregoing being illustrated by but not limited to substances such as antibodies, hormones, receptors, receptor binding molecules, antigens, drugs, toxicants, toxins, pathogens, biomarkers, biochemicals, cell surface markers, RNA, DNA and other molecules of interest.

More particularly, one embodiment of the present invention provides for methods for determining the presence, absence, or amount of a suspected analyte in a living mammal. The methods includes a first step of providing at least one affinity reagent “construct” having at least one Fc.epsilon.Rl receptor binding domain as defined herein and at least one additional moiety capable of binding the suspected analyte or other analyte of interest. The resulting reagent construct is capable of binding both a mast cell Fc.epsilon.Rl receptor and the analyte in any order and is considered multivalent (a.k.a. “multivalent affinity reagent construct”). The term “construct” is intended to mean the resulting structure or design of one single unit (i.e. molecule) of the affinity reagent of the present invention. The additional binding moiety may also be referred to herein as the “analyte binding moiety”. For purposes of the present disclosure, we note that determining the presence, absence or amount of a suspected analyte can be collectively thought of as analyzing for a suspected analyte.

For purposes of the present invention, it will be appreciated by those skilled in the art that the terms "affinity reagent", "AR", "reagent construct(s)", "affinity reagent construct(s)", multivalent affinity reagent", "multivalent reagent construct(s)", "reagent" and "construct(s)" are generally used and interpreted interchangeably and are each intended to refer to reagent(s) constructed in accordance with the present disclosure taken in conjunction with the examples and references incorporated herein.

It will also be appreciated by those skilled in the art that the descriptor “multivalent” is intended to mean that the reagent construct is capable of being bound to more than one entity at the same time. For purposes of the present disclosure, the term entity is intended to mean any biological or chemical substance or structure. For example, with respect to the multivalent affinity reagent construct of the present invention, “multivalent” is exemplified in that the affinity reagent construct is capable of being bound to at least one first entity (e.g. a mast cell Fc.epsilon.Rl receptor) at a first region of the construct (e.g. the Fc.epsilon.Rl receptor binding domain) and at least one second entity (e.g. the analyte of interest) at a second region of the construct by way of at least one additional moiety. Stated alternatively, the affinity reagent construct includes at least one first binding specificity to the mast cell Fc.epsilon.Rl receptor and at least one second binding specificity to an analyte of interest.

In accordance with the present invention, the dualistic binding discussed above is of course considered but one example of multivalent binding. It is responsible for the multivalent binding response of the present invention which occurs under crosslinking conditions as discussed below. The multivalent binding of the reagent construct is not necessarily order dependent and importantly, as further explained herein, the use of the present invention for sentinel monitoring contemplates that aspect. Further to that end, the analyte binding moiety itself may also, depending upon the type of moiety selected, possess multiple binding sites (i.e. multiple binding specificities) thereby imparting a monovalent, bivalent or polyvalent binding capability to the moiety depending upon the number of binding sites.

The methods contemplated by the present invention further include the step of contacting at least one endogenous mast cell in situ and present within a target tissue of a living mammalian subject with the affinity reagent construct of the present invention to elicit a multivalent binding response when the analyte of interest is or becomes present. It will be appreciated by the artisan that the aforementioned step is accomplished from a practical perspective by the physical delivery of a plurality of affinity reagent constructs (hereinafter such plurality simply referred to as the “affinity reagent” or “AR” ) to the target tissue by way of some device as discussed below. For purposes of the present invention, the term “target tissue” will mean the type of tissue to receive the affinity reagent of the present invention such as the skin.

In practice, the present invention contemplates selecting within a test subject an anatomical area and type of tissue having a localized concentration of endogenous mast cells that should range from about 125 to 20,000 mast cells per cubic millimeter for the species of mammal to which the affinity reagent is administered. For most mammals, the skin is an acceptable target tissue and more specifically, the upper dermal layer of the skin. For purposes of the present disclosure, the term skin is intended to encompass the superficial epidermal layer made of epithelial tissue, the deeper dermal layer or “dermis” made of connective tissue and lymph vessels, and the even deeper layer of fatty tissue typically referred to as the hypodermis. Mast cells are typically found in highest concentration immediately beneath the epithelial surfaces of the skin and the mucosa. In humans, delivery sites include the dermal layer of the upper arm or forearm and as well as those skin layers in lower body extremities (Janssens, et al., Journal of Clinical Pathology 58:285 (2005)). As will be further appreciated by the artisan from the passages below, the methods of the present invention can be said to comprise a bioassay in the sense that the underlying test is, in effect, being run or performed in vivo unlike traditional diagnostic assays.

It will be further appreciated that the terms “analyte”, “suspect analyte”, “suspected analyte” and “analyte of interest” shall be used and interpreted interchangeably for purposes of the present disclosure. The term “analyte” is intended to include chemicals, peptides, proteins, lipids, carbohydrates, glycoproteins, nucleic acid sequences, or a combination thereof, the foregoing being illustrated by but not limited to substances such as antibodies, hormones, receptors, receptor binding molecules, antigens, drugs, toxicants, toxins, pathogens, biomarkers, biochemicals, cell surface markers, RNA, DNA and other molecules of interest. Analyte is also intended to mean an analyte as defined herein, the presence, absence, or amount of which is known to be or suspected to be 1) an indicator of a disease, disease state or exposure to a substance or, alternatively, 2) an indicator of a subject’s general health, fitness, or emotional and/or physical state of being.

Disease and disease state fall under the broad category of a “pathology” and they can be generally defined as any harmful deviation from the normal structural or functional state of an organism and typically result in or are hallmarked by certain signs and symptoms but are different in etiology from physically induced injury. For example, an organism suffering from disease or a disease state commonly exhibits signs or symptoms that are abnormal relative to its normal, disease free state. Such signs and symptoms include the presence or absence of certain substances or, alternatively abnormally low or abnormally high levels of certain substances, all of such substances being potential analytes of interest that may be indicative of diseases and disease states for purposes of the present invention.

The terms “body fluid” or “bodily fluid” are intended to mean any fluid that originates from inside a mammalian subject including secretory, excretory or other flowable substances emitted from or contained within a subject such as blood, plasma, serum, lymph, interstitial fluid, intracellular or extracellular fluid, tears, saliva, urine, etc. The term “mast cell” is intended to mean basophils, eosinophils and any other cells that possess Fc.epsilon.Rl receptors. Moreover, the terms “Fc.epsilon.Rl receptor” and “high affinity receptor” are used interchangeably and are intended to mean the endogenous mast cell receptor for the Fc region of immunoglobulin E (IgE), while the term Fc.epsilon.Rl receptor binding domain represents any molecule(s) or entity capable of binding to the mast cell Fc.epsilon.Rl receptor whether generated from hybridoma technologies, recombinantly, chemically synthesized from amino acids or synthetically generated aptamers synthetically produced.

The method further includes performing an assessment of the multivalent binding response to determine the presence or amount of the analyte of interest. The multivalent binding response of the present invention is precipitated by the multivalent binding discussed above and encompasses physiological changes to the target tissue such as crosslinking of Fc.epsilon.Rl receptors across the mast cell membrane along with degranulation of the mast cell as further described below. Assessment of the multivalent binding response, if and when it that occurs, may be accomplished by various qualitative and quantitative approaches including a basic evaluation of the target tissue at or near the delivery site by measuring at least one physiological change in the target tissue or by visual inspection to identify at least one physiological change in the target tissue resulting from the binding response. Alternatively, a device capable of measuring at least one physiological change in the target tissue that is associated with the binding response may be employed.

The term “physiological change” is intended to mean morphological and biochemical differences that occur in mammalian tissue. For purposes of the present invention, a physiological change within or to the target tissue would include crosslinking of Fc.epsilon.Rl receptors across the mast cell membrane in mast cell(s) of the target tissue that are considered “localized” in proximity to the site of affinity reagent administration as well as degranulation of such mast cell(s) and any additional and secondary physiological changes to the target tissue that directly or indirectly result from such degranulation. The term “localized” is intended to mean the area starting from the area of contact in or on the target tissue being contacted by the device that delivers the affinity reagent up to an area radiating outward from such point of contact about 3 cm.

Mast cell degranulation includes the mast cell release of cellular constituents (a.k.a. granules) as part of the degranulation process as well as the immediate release of stored chemical mediators followed by newly synthesized mediators. These mediators also have their own primary and secondary effect on cells in the target tissue. These physiological changes occurring within or to the target tissue generally occur together and are very often inextricably intertwined. For example, some physiological changes result in a cascade of events that induce subsequent physiological changes such as a morphological change in tissue that is clearly visible by the eye. These changes can typically be evaluated either qualitatively or quantitatively.

As mentioned above, assessment of the binding response may involve performing a visual inspection to identify or measure a physiological change in the target tissue where the physiological change is, for example, the physical presence of swelling and erythema characteristic of a wheal and flare reaction induced by the biochemical changes that occur pursuant to degranulation. In another embodiment, assessment of the binding response may involve measuring a mast cell degranulation substance released by a mast cell during mast cell degranulation or alternatively measuring an additional and secondary substance that is released from cells in the target tissue in response to the degranulation substance released during degranulation. Alternatively, in yet another embodiment, assessment may involve using a device such as a biosensor or other apparatus to identify or measure a physiological change in the target tissue.

As mentioned above, the present method contemplates one embodiment wherein the affinity reagent is delivered into the skin, and optimally, into the dermis where the concentration of mast cells is at its highest. Generally, any tissue wherein the affinity reagent is exposed to from about 125 to 20,000 mast cells per cubic millimeter is suitable and is exemplified in various publications accessible to those skilled in the art. Delivery of the affinity reagent may be performed by any suitable means such as a needle prick, intradermal injection, solid needle, hollow needle, patch method or a needleless system.

Once familiar with the concepts of the present invention, construction of a multivalent affinity reagent may be accomplished using well known and established laboratory techniques including hybridoma technology, covalent conjugation, non-covalent binding, genetic engineering methods and related recombinant techniques among others, examples of which can be found in the cited references and other pertinent related art. The additional moiety or analyte binding moiety can be fashioned using at least one member from the group consisting essentially of antibodies, recombinant antibodies, engineered antibodies, antibody fragments, synthetic antibodies, engineered non-antibody binding proteins, antigens, chimeric molecules, fusion proteins, aptamers, hormones, receptors, receptor binding molecules, drugs, toxicants, toxins, pathogens, pathogen components, biomarkers, cell surface markers, ligands, RNA, or DNA as well as other chemical or biologic molecules to the extent they can impart the requisite analyte specificity to the bioassay as taught by the present invention. For example, in one embodiment the moiety is fashioned from an antigen such as hepatitis B surface antigen (HBsAg) or an antibody such as anti-hepatitis B surface antigen antibody (HBsAb) or anti-hepatitis B core antigen antibody (HBcAb). In an alternative embodiment, it can be fashioned from a protein such as interferon-gamma or troponin. In yet a further embodiment, the moiety may be fashioned from a non-infectious component of a pathogen such as lipoarabinomannan or a non-infectious version of a pathogen itself such as Ebola or Zika virus. These are but a few limited examples.

Additionally, the methods and kit of the present invention provide a method of testing for a suspected pathology in a living mammal. For purposes of the present invention, a suspected pathology can be defined as a particular disease or disease state thought to be present in the test subject. It will be appreciated that one must select the appropriate analyte of interest known to be or suspected to be an indicator of such disease or disease states and thereafter constructing an affinity reagent having at least one Fc.epsilon.Rl receptor binding domain and at least one additional moiety capable of binding the selected analyte wherein the resulting reagent is capable of binding both a mast cell and the analyte in any order; contacting a plurality of endogenous mast cells present within a target tissue of the mammal with the resulting reagent to elicit a multivalent binding response when the analyte is present; and performing an assessment of the multivalent binding response to determine the presence or amount of the selected analyte.

The methods and kit of the present invention also provide a method of testing a living mammal for exposure to a particular substance such as a chemical, drug, biowarfare agent, toxicant, toxin or pathogen. It should be noted that for purposes of the present application, the term “chemical” is intended to include pollutants such as toxic metals, polycyclic aromatic hydrocarbons, benzene, particulate matter (PM), nitrogen oxides, sulfur oxides, carbon monoxide, ozone and similar deleterious compounds. The method includes selecting an analyte known to be or suspected to be indicative of exposure to the particular substance and thereafter constructing an affinity reagent comprising at least one Fc.epsilon.Rl receptor binding domain and at least one additional moiety capable of binding the selected analyte wherein the resulting reagent is capable of binding both a mast cell and the analyte in any order; contacting a plurality of endogenous mast cells present within a target tissue of the mammal with the reagent to elicit a multivalent binding response when the analyte is present; and performing an assessment of the multivalent binding response to determine the presence or amount of the selected analyte.

Additionally, the methods and kit of the present invention provide a method of monitoring a living mammal for exposure to a particular substance such as a chemical, drug, biowarfare agent, toxicant, toxin or pathogen, or alternatively, a suspected pathology resulting from such exposure. The method includes selecting an analyte indicative of exposure to the particular substance and thereafter constructing an affinity reagent comprising at least one Fc.epsilon.Rl receptor binding domain and at least one additional moiety capable of binding the selected analyte wherein the resulting reagent is capable of binding both a mast cell and the analyte in any order; contacting a plurality of endogenous mast cells present within a target tissue of the mammal with the reagent for enabling a multivalent binding response when the analyte becomes present; and performing an assessment of the multivalent binding response, if it occurs, to determine the presence or amount of the selected analyte.

Additionally, the methods and kit of the present invention provide a method of testing as well as monitoring the health status a living mammal. The method includes selecting an analyte related to a health status parameter of interest and thereafter constructing an affinity reagent comprising at least one Fc.epsilon.Rl receptor binding domain and at least one additional moiety capable of binding the selected analyte wherein the resulting reagent is capable of binding both a mast cell and the analyte in any order; contacting a plurality of endogenous mast cells present within a target tissue of the mammal with the reagent to elicit or enable a multivalent binding response when the analyte is or becomes present; and performing an assessment of the multivalent binding response to determine the presence or amount of the selected analyte.

Moreover, the methods and kit of the present invention provide a method of monitoring or testing the fitness status a living mammal. The method includes selecting an analyte related to a fitness status parameter of interest and thereafter constructing an affinity reagent comprising at least one Fc.epsilon.Rl receptor binding domain and at least one additional moiety capable of binding the selected analyte wherein the resulting reagent is capable of binding both a mast cell and the analyte in any order; contacting a plurality of endogenous mast cells present within a target tissue of the mammal with the reagent for enabling a multivalent binding response when the analyte is or becomes present; and performing an assessment of the multivalent binding response to determine the presence or amount of the selected analyte.

Further, the methods and kit of the present invention provide a method of monitoring or testing a living mammal for a physiological change precipitated by exposure to environmental stress or injury such as a release of an endogenous biomarker in soldier embroiled in a battlefield firefight. The method includes selecting an analyte indicative of the physiological change to be detected, the choice of which should be a function of the environmental stress to which the test subject has been or will be exposed. The method further involves constructing an affinity reagent that includes at least one Fc.epsilon.Rl receptor binding domain and at least one additional moiety capable of binding the selected analyte wherein the resulting reagent is capable of binding both a mast cell and the selected analyte in any order; contacting a plurality of endogenous mast cells present within a target tissue of the mammal with the reagent for enabling a multivalent binding response when the when the analyte is or becomes present; and performing an assessment of the multivalent binding response to determine the presence or amount of the selected analyte. It will be appreciated that there are a wide array of environmental stressors including but not limited to extremes in temperature, fire, radiation, terrorist events, warfare, battle fatigue and climatic stressors as well as biologic or chemical stressors all of which induce certain physiological changes.

The present invention also provides for an affinity reagent for use in an in vivo bioassay used to detect an analyte wherein the reagent includes at least one Fc.epsilon.Rl receptor binding domain and at least one additional moiety capable of binding the analyte. The reagent is capable of binding both a mast cell and the analyte in any order.

Lastly, the present invention provides for a kit for performing an in vivo bioassay to determine the presence, absence, or amount of an analyte in a living mammal. The kit generally includes an affinity reagent that has at least one Fc.epsilon.Rl receptor binding domain and at least one additional moiety capable of binding the analyte. The reagent is capable of binding both a mast cell and the analyte in any order. The kit also includes a delivery device for delivering the affinity reagent to a target tissue of the mammal.

### BRIEF DESCRIPTION OF THE DRAWINGS

FIG. 1 shows schematic of a variety of affinity reagents to further exemplify the present invention.

FIG. 2 shows a basic approach for the delivery of the affinity reagent of the present invention to a target tissue followed by detection of a multivalent binding response.

### DETAILED DESCRIPTION OF THE INVENTION

While the methods and affinity reagents of the present invention may vary from one another in some aspects, the general approach and procedural steps set forth herein are all designed to determine the presence, absence, or amount of an analyte of interest in a mammalian subject. At the outset, the artisan will appreciate that in order to practice the methods and bioassay kit of the present invention, an affinity reagent (AR) must be constructed based upon the analyte of interest as further explained herein. Such analytes will broadly include a broad spectrum of substances and are intended to include chemicals, peptides, proteins, lipids, carbohydrates, glycoproteins, nucleic acid sequences, or a combination thereof, the foregoing being illustrated by but not limited to substances such as antibodies, hormones, receptors, receptor binding molecules, antigens, drugs, toxicants, toxins, pathogens, biomarkers, biochemicals, cell surface markers, RNA, DNA and other molecules of interest.

It will be appreciated that one must select the appropriate analyte of interest as an indicator of diseases or disease states as well as the detection and sentinel monitoring of certain environmental and conflict based exposures that may result due to bio-warfare and terrorist events. Similarly, selecting the appropriate analyte of interest is also important to determining the absence, presence or amount of a substance such as hormones, growth factors, metabolites, etc. as an indication of a subject’s general health, fitness or emotional and/or physical state of being.

Generally, diseases and disease state as well as exposures to undesirable substances are typically hallmarked by the presence of some associated analyte, the presence or absence of which may be used an indicator of the same. For example, such an analyte may be a pathogen, a component of or by-product produced by a pathogen. Such an analyte may also be a biomarker representing a pathogen’s presence in the subject to be screened or monitored. Alternatively, such an analyte may be endogenously released by cells of the subject as a direct or indirect response to the presence of a pathogen or an acute or chronic disease state such as TB. Such analytes can also be present due to certain genetically inherited pathologies or latent conditions that develop over time. There are also analytes that can be used to indicate the physical health, emotional health or fitness status of a subject. Exposure of a subject to certain chemicals and other substances may also result in the presence or absence of certain analytes in the subject, whether it is those the substances themselves or some analyte endogenously produced and released in the subject as a response to such conditions or exposures. Accordingly, the artisan will appreciate that when practicing the present invention, the selection of the appropriate analyte or analytes will be dependent upon the purpose of the testing sought to be performed. Those skilled in the art will also appreciate that there are numerous resources readily available and well known in the arts of physiology, medicine, epidemiology and related health sciences that may serve as guides when attempting to select an appropriate analyte.

As a footnote to the foregoing, the artisan will appreciate that in cases where the present invention will be used for sentinel monitoring, it is intended that while the AR delivered into the subject will react with the mast cells in the target tissue at the site of affinity reagent administration, crosslinking across the mast cell membrane will not occur unless the analyte is present. Once binding of the AR to the Fc.epsilon.Rl receptors on the mast cells has occurred, mast cells are “sensitized” to the analyte and are incorporated by the present invention to signal the presence of the analyte in the subject, typically pursuant to some event such as an exposure of the subject to a pathogen, toxin or biowarfare agent or a change in the subject’s physiology such as a biomarker that is normally not present or actively circulating in the blood, interstitial fluid, or the lymphatic system or is merely circulating at negligible levels. Upon the arrival of the analyte of interest in the bodily fluids of the subject at the AR delivery site, analyte binding to the AR occurs. This induces the multivalent binding response.

Once an appropriate analyte is selected, an affinity reagent (AR) for binding such analyte should be constructed. As will be appreciated from the discussion below, the analyte specificity of the test is determined by the additional moiety. Nonetheless, before engaging in construction of the reagent, it would be beneficial to have the artisan become more familiar with the pertinent terminology and the underlying mechanisms upon which the present invention is based. Accordingly, the following section has been provided for the convenience of the reader although the information is readily ascertainable from a myriad of resources currently available and well known in the arts of immunology, molecular biology and biochemistry.

In immediate (type 1) hypersensitivity, B-cells are stimulated (by CD4+TH2 cells) to produce IgE antibodies specific to antigens such as classic allergens and certain parasites. The difference between a normal infectious immune response and a type 1 hypersensitivity response is that in type 1 hypersensitivity, the antibody is IgE instead of the IgA, IgG, or IgM produced by B-cells. IgE antibodies bind to what is known as the Fc.epsilon.Rl receptor on the surface of tissue mast cells. This receptor is sometimes referred to as a “high affinity” mast cell receptor. Once these mast cells become bound to IgE antibodies, the mast cells are said to be “sensitized”. Subsequent exposure of the sensitized mast cells to the same antigen induces cross-linking of the IgE bound to those sensitized mast cells, resulting in anaphylactic degranulation. This degranulation is characterized by the immediate and explosive release of pharmacologically active pre-formed mediators from storage granules present in the mast cells as well as the concurrent synthesis of inflammatory lipid mediators from arachidonic acid. Some of these mediators include histamine, leukotriene (LTC4 and LTD4), and prostaglandin, which act on proteins (e.g., G-protein coupled receptors) located on surrounding tissues. The principal effects of these products are vasodilation and smooth-muscle contraction. Typically, a wheal (edema)-and-flare (erythema) reaction appears at the site of the surrounding tissues that is often referred to as hives. As mentioned above in the summary of the invention, the term “mast cell” is intended to mean any cells that possess Fc.epsilon.Rl receptor on their plasma membranes one exemplification being basophils and eosinophils to the extent they possess such Fc.epsilon.Rl receptors.

More particularly, the plasma membrane of mast cells is endowed with receptors that bind to the Fc portion of the IgE molecule at what is known as the “Fc.epsilon.Rl receptor binding domain”. These receptors bind circulating IgE with very high affinity and retain it at the mast cell surface for extended periods of time. Activation of the mast cell results by way of cross linking between bound IgE molecules, thereby inducing the fusion of the granules with the cell surface membrane. This leads to the exocytosis of the granule contents and the onset of an immunological cascade of events typically associated with allergies. (Aalberse, R.C., J. Allergy Clin. Immunol. 106:228 (2000); Al-Muhsen et al., CMAJ 168(10):1279 (2003); Scholl et al., The J. Immunol 175:6645 (2005); Handlogten et al., J. Immunol. 192:2035 (2014); Handlogten et al., Chemistry & Biology 21(20):1445 (2014); Matsuo et al., Allergology International 64:332 (2015)).

In humans and other mammals, mast cells are found in highest concentration immediately beneath the epithelial surfaces of the skin and the mucosa. Their location at the host-environment interface suggests a central role for these cells in immune surveillance that has been supported by numerous studies examining the responses of mast cells to various pathogens. One of the earliest observations about mast cells, made by their discoverer, Paul Ehrlich, is that they frequently adopt a perivascular localization within tissues. Mast cells are also in close proximity to lymphatic vessels in connective tissue (Ovary, Z., Jpn. J. Allergol. 43 1375 (1994); Turner et al., Nature 402 B24 (1999); Marcelino da Silva et al., Journal of Histochemistry and Cytochemistry 62 698 (2014)).

### Constructing the Affinity Reagent

The affinity reagent construct for the methods and bioassay kit of the present invention is comprised of at least one Fc.epsilon.Rl receptor binding domain and at least one additional moiety selected and fashioned for specifically binding to an analyte of interest. As mentioned above, the additional moiety or “analyte binding moiety” determines the analyte binding specificity of the present invention while the Fc.epsilon.Rl receptor binding domain binds the AR to the mast cell Fc.epsilon.Rl receptor. As mentioned above in the summary of the invention, this dualistic binding can also be referred to as “multivalent binding” and is responsible for what is termed herein as the “multivalent binding response” that occurs in the target tissue. The multivalent binding response occurs in the target tissue starting with the crosslinking at the Fc.epsilon.Rl receptors present on the mast cell(s) and leads to mast cell activation and other physiological changes or responses in the test subject that can be detected or measured.

Generally speaking, any molecule having analyte binding specificity to the analyte of interest can be fashioned to be incorporated into the reagent construct as an additional moiety during the construction of the affinity reagent of the present invention. For purposes of the present disclosure, the term “fashioning” or “fashioned” is intended to mean the preparation of the analyte binding moiety for incorporation into the reagent construct of the present invention and includes but is not limited to the removal of any extraneous materials unnecessary for incorporating the binding moiety into the reagent construct or for binding to the analyte of interest in order to leave intact the desired analyte binding specificity of the moiety. For example, a protein antigen may only require that portion of the molecule (i.e., a short amino acid sequence) necessary for the AR to bind the antibody of interest. Limiting unnecessary material can generally help to reduce cross reactivity of the AR. Fashioning or fashioned may also include the addition of materials not typically present on the analyte binding moiety but necessary for construction of the AR. For example, the addition of a linker to a drug may be required for the chemical conjugation of the drug to the F1.epsilon.R1 binding domain.

Some examples of suitable types of additional moieties used for the construction of affinity reagent of the present invention include antibodies, recombinant antibodies, engineered antibodies, antibody fragments, synthetic antibodies, engineered non-antibody binding proteins, antigens, chimeric molecules, fusion proteins, aptamers, hormones, receptors, receptor binding molecules, drugs, toxicants, toxins, pathogens, pathogen components, biomarkers, cell surface markers, ligands, RNA and DNA as well as biochemicals and chemicals that bind specifically substances. It will be appreciated by those skilled in the art that the moiety selected will be compatible in type and specificity to bind the analyte of interest.

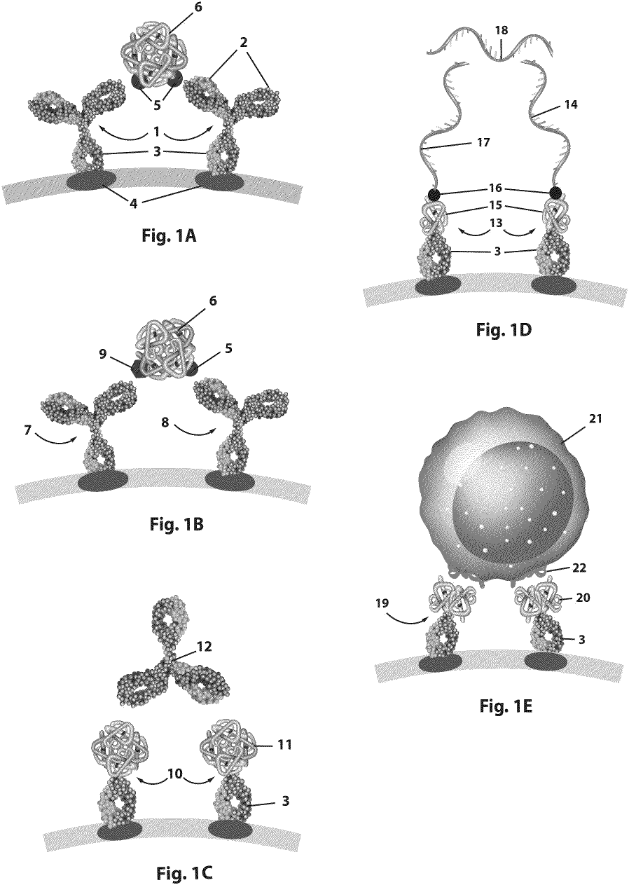

Turning now to the drawings, FIG. 1A shows a homogeneous population of AR molecules 1, both possessing antibody F(ab)₂ fragment 2 fashioned to serve as the additional moiety and an Fc-epsilon-R1 receptor binding domain 3 shown bound to a mast cell Fc.epsilon-R1 receptors 4. Those skilled in the art will readily appreciate that each arm of the antigen-binding fragment (Fab)₂ 2 is capable, in turn, of binding identical epitopes 5 on the antigen 6. Mast cell activation is accomplished through the binding of an analyte to at least two of the mast cell-bound AR molecules. FIG. 1B shows a heterogenous population of AR molecules where a first AR molecule 7 and a second AR molecule 8 have (Fab)₂ fragments that bind to a first epitope 5 and a different second epitope 9 on antigen 6, respectively.

Alternatively, FIG. 1C shows a homogeneous population of AR molecules 10, both comprising an Fc.epsilon.Rl receptor binding domain 3 and an antigen molecule 11 fashioned to serve as the additional binding moiety. The analyte of interest is an antibody 12. Mast cell activation is accomplished through the binding of at least two mast cell-bound AR molecules by the (Fab)₂ region of the antibody upon crosslinking.

FIG. 1D shows an AR molecule 13 comprising an Fc.epsilon.Rl receptor binding domain 3 and a single stranded DNA or RNA molecule 14 fashioned to serve as the additional binding moiety. In this example, the AR is constructed by covalently attaching avidin 15 to the Fc.epsilon.Rl receptor binding domain 3 which is then allowed to react with a biotin-labeled 16 nucleic acid molecule 17, thereby, rendering the AR molecule. Avidin-biotin forms one of the strongest non-covalent bonds known and is commonly used for the purpose of attaching one molecule to another. Those skilled in the art will appreciate that the avidin-biotin model is exemplary only and various other methods of attachment are suitable for the present invention. It will be further appreciated that the method contemplated to incorporate the AR as exemplified here is designed to detect circulating DNA or RNA 18 having a complimentary nucleic acid sequence to the DNA or RNA of the AR molecule.

In FIG. 1E, the AR molecule 19 is shown comprised of an Fc.Epsilon.R1 receptor binding domain 3 and a TC-cell receptor binding protein 20 to serve as the additional binding moiety. When T-cell 21 comes in close proximity to the sensitized mast cell, the T-cell binds the AR through a cell-surface receptor 22 and activation occurs upon crosslinking. Similar to the example shown in FIG. 1E, viruses and bacteria recognize their hosts through receptor binding molecules by binding to a specific receptor on the host cell surface. An AR comprising a receptor binding molecule to serve as the additional moiety can also be used to directly detect viruses or bacteria. For example, an AR molecule wherein the analyte binding moiety is composed of a CD4 or CD48 (or TLR2) receptor could be used to detect HIV virus or mycobacterium tuberculosis bacteria, respectively. Likewise, an AR molecule wherein the analyte binding moiety is composed of the angiotensin-converting enzyme 2 (ACE2) receptor (active or inactivated) could be used to detect the SARS-CoV-2 virus.

Importantly, the drawings are exemplary of only a limited number of embodiments for constructing the affinity reagent of the present invention. The analyte binding moiety of the present invention may be comprised of a peptide, protein, nucleic acid, lipid, carbohydrate or any combination thereof, for example. Moreover, the AR may be either homogeneous wherein the AR molecules that are delivered to the target tissue are composed of identical AR molecules or a heterogeneous combination of AR molecules that recognize different binding sites on the analyte of interest. Furthermore, the analyte binding moiety of the AR may be mono-specific requiring the analyte to possess two or more identical epitopes or binding sites for cross-linking. Alternatively, the binding moiety may be bi-specific, poly-specific, or a mixed population of ARs allowing crosslinking through binding of similar and dissimilar epitopes or binding sites on the analyte of interest. Importantly, those skilled in the art will readily appreciate that the AR repertoire for each analyte can be constructed in a myriad of ways to optimize performance of the assay and to recognize analyte variants or isotypes.

There are a multitude of available in vitro techniques and recombinant methods that the artisan can employ to construct an Fc.epsilon.Rl receptor binding domain with an analyte binding moiety to form the AR as contemplated pursuant to the present invention, examples of which include hybridoma techniques, covalent conjugation, non-covalent binding, genetic engineering methods, recombinant techniques, or a combination thereof. Genetic engineering methods, for example, employ recombinant DNA methods to form proteins not normally produced by cells and provide an avenue for those proteins to be expressed in large quantities in a variety of expression vectors. For purposes of the present invention, the terms “in vitro construction” and “constructing in vitro” are intended to have the ordinary and customary meanings attributed to them by those skilled in the art. Similarly, the terms “recombinant construction” and “constructing recombinantly” are also intended to have the ordinary and customary meanings attributed to them by those skilled in the art.

Additionally, the artisan will take note that the Fc.epsilon.Rl receptor binding domain of human IgE comprising C2, C3, and C4 regions is sufficient for binding to alpha chain of the high affinity receptor (Fc-epsilon-R1) mast cell receptor. Studies with human IgE Fc domains have demonstrated that the smallest fragment that shows Fc-epsilon-R1 binding activity spans amino acids 329-547 and lacks the entire C2 domain. Moreover, studies have also shown that Fc fragment 315-547 is an S-S-linked dimer and that Fc fragment 329-547 that forms a dimer without S-S bonding, both bind the Fc-epsilon-R1 receptor with high affinity. The presence of N-linked sugars did not appear to be necessary for high affinity binding (Basu et al., The Journal of Biological Chemistry 268:13118 (1993)). The Fc.epsilon.Rl receptor binding domain can be purified directly from proteolytic digest of IgE molecules or can be produced by recombinant methods. The Fc.epsilon.Rl receptor binding domain from IgE has been recombinantly produced and purified from a variety of expression vectors (Kamiya et al., Exp. Med. 180:297 (1996); Liu et al., Proc. Natl. Acad. Sci. USA 81:5369 (1984)). As such, construction of the Fc.epsilon.Rl receptor domain of the AR will typically include the C2, C3, and C4 regions. In an alternative embodiment, such construction will include at least the C3 and C4 regions, and at a minimum, amino acids 329-547.

Those familiar with the art will appreciate that Fc.epsilon.Rl receptor binding domains derived or modified from any species, recombinantly synthesized or otherwise constructed will generally be sufficient to bind to mammalian F1.epsilon.R1 mast cell receptors. Accordingly, the methods and kit of present invention may be readily modified to test any mammal that possesses mast cells, examples of which include humans, dogs, cats, horses, farm animals, and livestock provided that the design and adaption of the methods and kit for each species integrate proper consideration regarding sensitivity, cross reactivity, etc. as between the different species for which a test is being developed. To that end, there are various humanization protocols available to address those situations where a moiety derived from another species may be modified for use in humans.

As mentioned above, one embodiment of the present invention comprises antibodies or antibody fragments that may be fashioned for use as the additional moiety. Those skilled in the art will readily appreciate that antibodies available in the art as contemplated for use with the present invention may be fashioned using standardized tools and techniques readily available for designing and developing research products, diagnostics and therapies. Generally, antibody molecules may be engineered to include monospecific, bispecific or tri-specific antigen binding domains as well as being further manipulated to render non-humanized, humanized or chimeric molecules. Whether utilized in whole or in part, such antibodies exemplify suitable analyte binding moieties that may be fashioned for the AR molecule. Over the past three decades antibodies have been dissected into smaller antigen binding fragments, initially by proteolysis and later by genetic engineering to produce mono or multivalent fragments that include, but not limited to, Fab, F(ab)₂, Monospecific Fab₂, Bispecific Fab₂, Trispecific Fab₃ fragments. Antibody fragments (e.g., single chain variable fragments (scFv), and V.sub.HH domains) and artificial affinity binders (e.g., Affibodies, Monobodies, DARPins, etc.) have been created and are developed by screening large gene libraries of potential binders with various panning technologies. Such antibody fragments also make suitable analyte binding moieties that may be fashioned for the AR molecule.

Additionally, various technologies have afforded the development of numerous protein scaffolds with unique affinity interaction domains that bind target epitopes (Groff et al, Biotechnology Advances 33:1787 (2015); Marx, V. Nature Methods 10(9):829 (2013). Antibody-antigen binding diversity can also be acquired by unorthodox mechanisms that represents a third layer of diversification of immune repertoires beyond the variability introduced by recombination and mutagenesis (as described by Kanyavuz et al., Nature Reviews, Immunology 19:355 DOI:org/10.1038/s41577-019-0126-7 (2019)). Furthermore, additional specificities may be acquired by using phage display technology which has been used extensively to generate large libraries of antibody fragments by exploiting the capability of bacteriophage to express and display biologically functional protein molecule on its surface. Combinatorial libraries of antibodies have been generated in bacteriophage lambda expression systems which may be screened as bacteriophage plaques or as colonies of lysogens (Marks et. al., Biotechnology 10 779 (1992). Accordingly, any of the foregoing or derivatives thereof would constitute suitable analyte binding moieties for constructing the affinity reagent of the present invention.

Aptamers are similar to antibodies in that they can bind to proteins and modulate their function, and they are often referred to as chemical antibodies due to their synthetic production (Groff et al, Biotechnology Advances 33:1787 (2015); Marx, V. Nature Methods 10:829 (2013). Aptamers also make suitable analyte binding moieties for the AR molecule. Aptamers are short, single-stranded DNA or RNA oligonucleotides that can bind to their targets with high specificity and affinity through van der Waals forces, hydrogen bonding, salt bridges, and hydrophobic and other electrostatic interactions. Aptamers have the ability to fold into complex and stable three-dimensional shapes, which allows them to fold within or around their targets. DNA aptamers have greater chemical stability, while RNA aptamers produce more structural shapes due to their greater flexibility. Aptamers have been identified against targets that include small organic and inorganic molecules, such as dyes, nucleotides, amino acids, and drugs; biopolymers, such as peptides, proteins, and polysaccharides; ions; phospholipids; nucleic acids; viruses; bacteria; cell fragments; and whole cells. Accordingly, aptamers are further examples of analyte binding moieties that can be incorporated into the AR of the present invention.

There are a number of molecules that can participate in molecular binding and include proteins, nucleic acids, carbohydrates, lipids, and small organic molecules such as drugs. Types of complexes that form as a result of molecular binding include: protein-protein, protein-DNA, protein-hormone, and protein-drug. These include proteins that form stable complexes with other molecules. Cellular receptors are structures composed of protein that receive and transduce signals which are typically integrated into biological systems. These signals are typically chemical messengers, which bind to a receptor and cause some form of cellular/tissue response, e.g. a change in the electrical activity of a cell. A molecule that binds to a receptor is typically referred to as a ligand. Ligands can be a proteins or peptides, or other small molecules such as hormones, pharmaceutical drugs, toxins, or parts of the outside of a virus or microbe. Receptors and receptor ligands are both additional examples of analyte binding moieties that can be incorporated into AR molecules by those skilled in the art.

Circulating nucleic acids (CNA) have been reported in a number of clinical disorders like cancer, stroke, trauma, myocardial infarction, autoimmune disorders, and pregnancy-associated complications. The term CNAs refers to segments of genomic, mitochondrial or viral DNA, RNA and microRNA (miRNA) found in the bloodstream. Scientists have now discovered disease-specific genetic aberrations, such as mutations, microsatellite alterations, epigenetic modulations (including aberrant methylation), as well as viral DNA/RNA from nucleic acids in plasma and serum. CNA has received special attention because of its potential application as a non-invasive, rapid and sensitive tool for molecular diagnosis and monitoring of acute pathologies and the prenatal diagnosis of fetal genetic diseases (Suraj et al., Biomedical Reports 6:8 (2017). Adapting complimentary sequences of CNAs associated with diseases and disease states will provide yet another analyte binding moiety for construction of the AR of the present invention.

The analyte binding moieties described above can be fashioned and incorporated into the AR of the present invention by a number of methods well known in the art including covalent conjugation and non-covalent binding methods. Additionally, molecular techniques can be employed to construct and produce AR molecules as fusion proteins wherein the nucleic acid sequence of both the Fc.epsilon.Rl receptor binding domain and the analyte binding moiety are recombinantly joined and inserted into an expression vector. Fusion proteins or chimeric proteins (literally, made of parts from different sources) are proteins created through the joining of two or more genes that originally coded for separate proteins. Translation of this fusion gene results in a single or multiple polypeptides with functional properties derived from each of the original proteins. Recombinant fusion or chimeric proteins are created artificially by recombinant DNA technology for use in biological research or therapeutics. The terms chimeric or chimera usually designate hybrid proteins made of polypeptides having different functions or physicochemical patterns. Accordingly, both fusion proteins and other chimeric molecules also provide suitable choices to be incorporated as the additional binding moiety of the present invention.

Additionally, monoclonal and polyclonal IgG, IgM, IgA, and IgD antibodies can of course be sequenced and produced recombinantly, providing an easy pathway for isotype switching from Fc gamma, Fc mu, Fc alpha, and Fc delta to Fc epsilon, respectively. AR molecules can also be generated by in vitro methods using hybridoma technologies, essentially producing IgE molecules possessing the Fc.epsilon.Rl binding domain and F(ab)₂ region specific to the analyte of interest. These techniques are all well known in the art. Pathogens sometimes recognize host cells through receptor binding proteins such as CD4, CD48, ACE2 or TLR2. These receptors and other pathogen components may be used as the analyte binding moiety on affinity reagents in order to assess disease states. For example, the number of CD4 cells are routinely measured in patients with HIV as an indicator of advanced HIV disease.

In light of the foregoing, it will be readily appreciated by the artisan that one may readily modify and substitute one AR for another AR to create a different kit embodiment. Accordingly, it will be further appreciated that standardized manufacturing SOPs embracing the present invention can be quickly modified to allow the rapid production of a new AR molecules and novel bioassay kits thereby providing an extensive platform for diagnostic testing. New AR-bioassays can be carried through similar consistent product development cycles to yield rapid and large-scale deployment in foreseen and unforeseen healthcare emergencies. Patient implementation programs can be standardized as well, thereby reducing the uncertainty typically associated with the introduction of new methods.

### Contacting Mast Cells With AR

As mentioned above, the methods and kit of the present invention further include the step of sensitizing mast cells by contacting the affinity reagent in vivo with a plurality of endogenous mammalian mast cells via delivery of the AR to mast cells that are present within a target tissue of a living mammalian subject to elicit and enable a multivalent binding response when the analyte of interest is or becomes present. It will be appreciated by the artisan that the aforementioned delivery also contemplates selecting within a test subject an anatomical area and type of tissue having a concentration of endogenous mast cells ranging from about 125 to 20,000 mast cells per cubic millimeter for the species of mammal upon which the delivery is to be performed. For example, in most mammals, the skin is an acceptable target tissue and more specifically, the upper dermal layer of the skin. In humans, delivery sites include the skin of the upper arm or forearm and lower body extremities at approximately the same depth and manner as a typical allergy prick test or tuberculin skin test, the particulars of which are well known and established in the clinical arts. Notwithstanding the foregoing, any tissue wherein the affinity reagent is exposed to from about 125 to 20,000 mast cells per cubic millimeter would generally be suitable provided that the method for detecting the multivalent binding response can be effectively utilized and is reasonably practical for clinical purposes.

Physical delivery of the affinity reagent may be performed by any suitable means including but not limited to a needle prick, intradermal injection, solid needle, hollow needle, micro-needle, patch method or a needleless system. In one simple embodiment, the AR is delivered into the skin by a needle. The needle may be either solid or hollow. The AR is injected into the skin by hollow needle or the needle can be solid and coated with an AR coating, wherein the AR is released from the needle when the needle makes contact with bodily fluids.

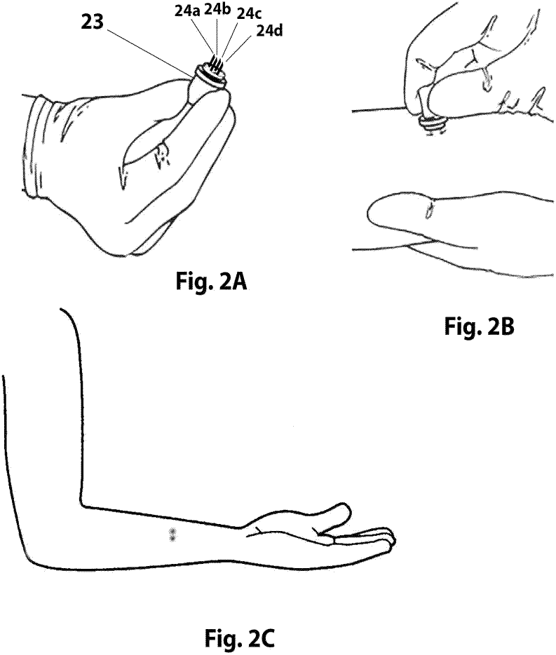

For example, FIG. 2A shows a multipronged applicator 23 with four solid needles 24a-24d that can be coated with four different AR molecules in a multiplexing format as discussed further below. By way of example only, one needle can be used for a positive control 24a. Another needle can be used for a negative control 24b. Yet another needle can be coated with an AR that recognizes an analyte 24c, while another needle can be coated with an AR that recognizes a different analyte 24d. A positive control can be, for example, an AR molecule having an analyte binding moiety with specificity to serum albumin or an endogenous molecule that is always systemically present and continuously circulating within the subject being tested. Additionally, a positive control may include histamine in order to make sure that the patient has a valid wheal and flare/erythema response. A negative control can be, for example, an AR molecule demonstrating binding activity to any substance not present in the species being tested. FIG. 2B shows the delivery device or applicator 23 being applied to and penetrating the skin, rehydrating and releasing the AR molecules coated on the delivery end of the device that may then freely bind to mast cells in situ in addition to circulating analyte present in the target tissue. As shown in FIG. 2C, the patient test positive for only one analyte and a positive control. The negative control and one analyte are negative with no visible response. The positive control and the other analyte are positive showing two wheal and flare reactions. In the example described above, the assay method described is used in multiplex. Multiplex assays measure multiple analytes in a single experiment. There are many ways to position or mark a multiplex test in order to track and confirm the position of each analyte or control.

Where applicable, the tests along with positive and negative controls can be applied together or separately and, in any order, and at any time. For example, when using commercially available or custom designed allergy skin test applicators, a single applicator having a single prong that includes one or more spike members to penetrate the skin can be used to apply the test as well as a positive control and a negative control sequentially as needed. Similarly, a dual prong applicator, with each prong having one or more spike members, can be used to apply the test and a control in parallel. In addition, a multiprong applicator with each prong having one or more spike members can be used to apply a variety of tests and controls in parallel. Each prong can also have a touch activator to reduce pain which is well known to practitioners who employ allergy testing devices in their practice.

Multiplex use of the invention provides a means of detecting an active disease signature, thereby preventing unnecessary testing and treatment. For example, instead of detecting antibodies from patients that have been previously exposed to HBV and have cleared the virus, this invention can be used to detect viral particles, DNA, RNA, and other constituents of the virus simultaneously without taking multiple samples in order to demonstrate the presence of active disease. (Liang, T.J., Hepatology 49(5 Suppl):S13-S21 (2009); Gerlich, W.H., Virology Journal 10(239) http://www.virologyj.com/content/10/⅟239 (2013); Venkatakrishnan et al., Annu. Rev. Virol. 3:451 (2016); Jiang et al., Journal of Virology 90(7):3330 (2016); Hu et al., Viruses 9 56 (2017)).

As shown below, in Table 1 in information from the Center for Disease Control, Atlanta, Ga, detecting both viral particles (HBsAg) and antibodies (HbsAb and HBcAb) at the same time using an AR applied in multiplex can provide a definitive diagnosis and treatment plan for hepatitis B patients which is not currently commercially available (Diepolder et al., Gastroenterology 116 650 (1999); Kimura et al., Journal of Clinical Micro. 41(5):1901 (2003); Tong, S., Int. J. Med. Sci. 2(1):2 (2005); y et al., Journal of Clinical Microbiology 44:2321 (2006); Gallagher et al., Virology 502:176 (2017)).

Table 1

Hepatitis B Test Panel

| | | | |
|---|---|---|---|
| Interpretation and Action Needed | HBsAg | HBsAb | HBcAb |
| Not Immune - Get Vaccinated | Neg | Neg | Neg |
| Immune - Protected | Neg | Pos | Pos |
| Immune - Protected | Neg | Pos | Neg |
| Infected - Need More Testing | Pos | Neg | Pos |
| Could be Infected - Need More Testing | Neg | Neg | Pos |

As discussed, the AR can be delivered into an area of the skin in situ (or other applicable mast cell rich areas), singularly or in multiplex, by needle prick or intradermal injection or patch method, including needleless systems. Recent advances in intradermal delivery needle design have reduced the pain associated with injections. Smaller gauge and sharper needles reduce tissue damage and therefore decrease the amount of inflammatory mediators released (for reference see Zehrung et al., “Intradermal Delivery of Vaccines: A review of the literature and the potential for development for use in low- and middle-income countries” Program for Appropriate Technology in Health (PATH) Aug. 27, 2009; Ita, K., Pharmaceutics 7:90 (2015); Larraneta et al., Materials Science and Engineering R 104:1 (2016); Martin et al., Safety 3:25 D01:10.3390/safety3040025 (2017); Shrestha et al., Scientific Reports 8:13749 DOI:10.1038/s41598-018-32026-9 (2018).

Microneedles are typically less than 0.2 mm in width and less than 2 mm in length. They are usually fabricated from silicon, plastic or metal and may be hollow for delivery or sampling of substances through a lumen or the needles may be solid and coated with substances. By selecting an appropriate needle length, the depth of penetration of the microneedle can be controlled to avoid the peripheral nerve net of the skin and reduce or eliminate the sensation of pain. The extremely small diameter of the microneedle and its sharpness also contribute to reduced sensation during the injection.

The advantages of needle-free injection devices have been recognized for some time. Some of the advantages of needle-free devices and methods include the absence of a needle which can intimidate a patient and also present a hazard to healthcare workers. The injection jet generated by a needle-free device is generally smaller in diameter than a hypodermic needle and thus, in certain instances, a needle-free injection is less painful than an injection provided by a hypodermic needle device. Because of these and other advantages of needle-free injection many variations of pneumatic, electronic or spring activated needle-free injection devices have been designed to provide a single injection, or alternatively a series of injections to one or more patients. Most known needle-free injection devices operate by driving the injectable fluid through a nozzle with a powered piston to create a fine, high-pressure jet of fluid that penetrates the skin.

Additionally, there are pre-fillable delivery devices available for administering intradermal injections which include a pre-fillable container adapted to store a substance. The substance is expelled through a needle cannula having a forward tip adapted to administer the intradermal injection. A limiter surrounds the needle cannula having a generally flat skin engaging surface extending in a plane generally perpendicular to the needle cannula. An insert is centrally located within the skin engaging surface and is pierceable by the forward tip. A sleeve with a first end and a second end surrounds the pre-fillable container. The limiter is inserted through the first end and the second end is affixed to a depressible plunger for expelling the substance from the container. The limiter is movable between a first position and a second position thereby exposing the forward tip and is selectively movable between a third position and a fourth position thereby concealing the forward tip. These and other devices are available for pain-free and safe application of substances intradermally and the described methods and others are well known to those knowledgeable in the art.

It will be appreciated by the artisan that the AR can be applied alone or together with any of several well known pharmaceutical carriers suitable as a vehicle for adding constituents that may be used to influence AR binding kinetics, promote chemotaxis, or elicit substances from near-by cells that can be evaluated and measured. The vehicle can be added simultaneously with the AR or separately.

It will be further appreciated that there are a number of techniques currently available in the clinical laboratory and medical manufacturing arts for rendering a suitable vehicle for carrying the AR into the target tissue or for depositing it onto the delivery end of the device until the device is utilized to deliver the AR into the target tissue. In one embodiment, AR may be suspended in a liquid medium of suitable viscosity that is used, for example, to deposit the AR onto the delivery end of the device such as the end of a solid needle whereupon it is subsequently dried to render an AR layer deposited on the device until used on a subject. Alternatively, the AR may, for example, be delivered to the target tissue in a liquid medium that is delivered by hollow needle or needless system and injected into the target tissue. The selection of such medium should integrate factors such as storage conditions and shelf life. Various suitable substances are commercially available for use as coating mediums or vehicles by which to carry or deposit the AR provided that they are properly selected for the AR to be delivered, the particulars of which are further addressed below.

The vehicle or medium for applying, transporting, storing, using, etc. the AR may be dependent on the AR construct itself. The vehicle may be liquid or dry and may include buffers, salts, protease or nuclease inhibitors, bacteriostats, chemotactic agents, detergents, stabilizing reagents, etc., or may be composed of proprietary reagents provided by a 3^{rd} party supplier. The list of additives is broad and well known to those practiced in the art. The AR is delivered in vivo and therefore resides in bodily fluids once delivered. It optimally performs within the physiologic pH range of 6 -9.

As mentioned above, the AR can be applied singularly without additional constituents or in a vehicle suitable for adding constituents that may be added to influence AR binding kinetics, promote chemotaxis, or may elicit substances from nearby cells that can be measured as contemplated by the methods of present invention. Moreover, the vehicle for delivery may be optimized to perturb the equilibrium between endogenous, preexisting IgE that is bound to mast cells and the newly available AR molecules in order to provide a method by which to increase the concentration of AR molecules on the mast cell. Equilibrium shifting towards AR binding may be accomplished by a variety of methods that are well known in the art. For example, the addition of excess AR, changes in pH, changes in salt concentration, and the addition of detergents may be employed for equilibrium shifting.

The delivery step of the methods of the present invention may also include a vehicle composed of a chemotactic or other constitutes that draw cells of interest to the target tissue. While the multivalent binding response can, in theory, be localized to many body regions, the most common site will consist of AR-sensitized mast cells located in interstitial fluid of the target tissue. This provides an opportunity to detect cells expressing specific cell surface markers such as distinct immune cell populations. Detection or measurement of receptor binding ligands such as LAG3 cleavage, CD8/Treg ratios, cytokines, chemokines can be used to select and manage patients on immunotherapy. Chemotactic factors can be introduced concurrently or synchronously with the AR to attract cells of interest to the target tissue.

The delivery vehicle may also allow the methods of the present invention to elicit and detect analytes secreted or released from surrounding cells. Delivery of the AR may optionally include a vehicle composed of constituents that cause release of analytes from neighboring cells, such as T-lymphocytes, known to be in close physical proximity to endogenous mast cells. The AR specific for the analyte released from the T-cells, would detect their presence. For example, Interferon-γ release assays (IGRA) are in vitro tests used in the diagnosis of some infectious diseases, especially tuberculosis. Interferon-γ (IFN-γ) release assays rely on the fact that T-lymphocytes will release IFN-γ when exposed to specific Mycobacterium tuberculosis (MTB) antigens. T-cells from most persons that have been infected with MTB will release INF-gamma when a patient’s sample is mixed with certain MTB antigens. Accordingly, MTB antigens could be delivered to the target tissue in addition to an AR specific for INF-gamma. MTB antigens included in the AR delivery medium would stimulate release of INF-gamma from adjacent pre-exposed T-cells. The INF-gamma could then be detected using a method wherein the AR binds to INF-gamma triggering mast cell degranulation. The presence of INF-gamma correlates to MTB infection. Additionally, the methods and kit of the present invention reduces the complexity of a test that is similar to the IGRA test but eliminates the need to remove a patient’s sample of blood and the necessity for performing the test on a benchtop under tissue culture conditions.

Further yet, there are numerous molecules released from mast cells during and after degranulation, any of which can be used to measure a response; in solo, in combination, or through enhanced secondary reactions and cascades. Accordingly, in another further embodiment of the invention, a probe placed in a target tissue may be retained there as a physicochemical detector. Biologically sensitive detectors may be created using biological engineering techniques known in the art. A transducer or the detector element deployed in the target tissue could be easily designed to transform a signal or change occurring as a function which occurs as a result of the multivalent binding response into a second signal for easy measurement and quantification. Alternatively, the delivery device may be used to deliver the AR to the target tissue while remaining positioned thereafter to operate as the detector as discussed above.

Delivery of active molecules into or via the skin (transdermal delivery) can offer significant advantages over the more conventional routes of delivery. The use of chemical penetration enhancers such as dimethylsulfoxide that interact with skin lipids to facilitate the transport of the AR across membranes into the dermal layer of the skin.

Dyes including but not limited to methylene blue, fluorescein and fluorescent peptides that can be detected visually or by device in the dermal layer of the skin may be used to enhance detection/assessment of the multivalent binding response of the present invention. For example, dyes such as methylene blue can be included in the application vehicle to provide an indicator that the affinity reagent was applied correctly to the dermal layer of the skin. There are many adaptations that can be envisioned where adding dyes or fluorescent compounds and other reagents to the application vehicle or AR that are visible by eye or detectable device could be utilized. Attaching a dye to the AR such a fluorescein would allow the monitoring of a positive reaction by way of an external fluorescent detector. Monitoring dyes can be used to determine parameters such as density of mast cells at the application site, the number of AR molecules occupying mast Fc.epsilon.Rl receptors and the rate of the reaction. Measurement of these parameters and others may be used singularly or combination to quantitatively or semi-quantitively measure analytes using the current invention.

### Assessing the Multivalent Binding Response

The methods of the present invention further include performing an assessment of the multivalent binding response to determine the presence, absence, or amount of the analyte of interest. It will be appreciated that assessment of such a binding response presupposes providing adequate time for a binding response to occur. Binding response times are dependent upon the purpose or intended reason for performing the method as well as the concentration of the analyte in the target tissue at the time the AR is delivered.

The multivalent binding response of the present invention is a function of the multivalent binding discussed above and encompasses crosslinking of Fc.epsilon.Rl receptors across the mast cell membrane along with degranulation of the mast cell and any additional and secondary physiological changes to the target tissue that directly or indirectly result from such degranulation. Assessment of the multivalent binding response, if and when it that occurs, may be accomplished by various qualitative and quantitative approaches including a basic clinical evaluation of the target tissue at or near the delivery site by visual inspection to identify at least one morphological change in the target tissue that is associated with the binding response (e.g. a wheal and flare dermal reaction). Alternatively, a device capable of measuring at least one changes in the target tissue that is associated with the binding response may be employed to evaluate the tissue.

Since there are numerous molecules released from mast cells during degranulation, any of these molecules may be theoretically used to measure a response whether in solo, in combination, or through enhanced secondary reactions and cascades. As such, performing an assessment of the binding response may be accomplished by measuring a biochemical change to the target tissue such as a mast cell degranulation substance released by mast cell degranulation or by measuring an endogenous substance released in response to a mast cell degranulation substance. Alternatively, a wheal and flare response is a physiological tissue change that is visible and easily measured. Devices may also be employed to measure parameters associated with a wheal and flare reaction such as width, density or intensity, tensile changes in skin, or the heat generated from a positive reaction. Moreover, enhancing technologies such as secondary reagents and conjugates thereof provides an additional secondary mode of signal enhancement and measurement.

A sensor or biosensor may also be employed to assess the binding response by way of transducers or similar apparatus. As mentioned earlier in the disclosure, a probe may be placed in proximity of the AR after delivery to a target tissue and retained there as a physicochemical detector. Biologically sensitive elements may be created using techniques generally known in the arts of biomedical engineering and related fields. A transducer or the detector element deployed in the target tissue could be easily designed to transform a signal or change occurring as a result of mast cell degranulation. Three major techniques are based on their transduction mechanism, namely electrochemical, optical, and acoustic-based sensors. Electrochemical sensors are further divided into impedimetric, amperometric, potentiometric, and conductance-based sensors. Optical sensors include fluorescence- and chemiluminescence-based biosensors, surface-enhanced Raman spectroscopy (SERS), and surface plasmon resonance (SPR)-based sensors. Acoustic wave-based sensors have two subcategories, quartz crystal microbalance (QCM)-based and surface acoustic wave (SAW)-based sensors. The sensor reader device with the associated electronics or signal processors would lead to a display of the results in a user-friendly way applicable to connectivity networks, smartphones and similar displays and communication devices as well as user interface mechanisms. Sensor technology is a rapidly developing field and there are many variations known to those experienced in the art that could be employed. The applicator can both introduce the AR to mast cells and can act as the detector element providing a single step assay and result (Upasham et al, Advanced Health Care Technologies 4:1 https://doi.org/10.2147/AHCT.S138543 (2018); Turner et al., Oxford, UK: Oxford University Press. p770 (1987); Banica et al., UK: John Wiley & Sons p576 ISBN 9781118354230 (2012); Dincer et al., Advanced Materials 31(30) doi:10.1002/adma.201806739 (2019).

In situations where the method is being used to test a population or group for suspected exposure to a substance such as a pathogen, toxin, etc., the expected binding response time will generally be either immediate or will occur within a few seconds provided that the test subject has been exposed to the substance and that the analyte selected to hallmark that exposure is actively circulating within the test subject. Generally, if the analyte is actively circulating, it can be expected to be found in the interstitial fluid at the AR delivery site within the target tissue. In contrast to the foregoing, there are situations where the method is being used as a sentinel test to perform an ongoing monitoring function to detect a future exposure as further described in the section below. As expected, a multivalent binding response will not occur until exposure to a substance has taken place and the analyte selected to hallmark such an exposure has had time to circulate to the target tissue delivery site. In short, performing an assessment of the multivalent binding response is secondary to providing a sufficient opportunity for a detectable multivalent binding response to occur in the test subject. Where the binding response is immediate, assessment may, in turn, be done immediately.

It will be further appreciated that the multivalent binding response time may also vary depending upon the analyte of interest to be detected and/or the character of the analyte binding moiety utilized for the reagent construct. Binding response times may be adjusted based upon the construction and concentration of the AR, the vehicle used for contacting the mast cells with AR, and the detection method. Notwithstanding the foregoing, adequate detection times may be generally categorized as follows: a rapid test method would range from immediately (T¹= 0) up to about two hours (T² = 2 hrs); a sensitive test method would range from two hours (T¹ = 2 hrs) to about 48 hrs (T² = 48 hrs); and the AR-sentinel test would have an expiration date of ranging from about 12 weeks to about 6 months from the time of delivery of the AR to the target tissue.

In the example sections below, binding responses and the resulting tissue change ranged from 30 sec to 8 hours depending on the concentration of HBsAg in the circulatory system. As an example of a sensitive method, mice challenged with 20 ug or more of HBsAg were positive after 20 min. However, mice challenged with 10 ug could only be detected after 8 hours. Unlike all blood assays, a bioassay performed by the methods of the present invention affords continuous exposure of the assay to the analyte as it circulates throughout the body. This continuous exposure enables very low detection levels and imparts significant sensitivity to the assay. Accordingly, it will be appreciated that bioassays incorporating the methods of the present invention are generally not limited by the amount of analyte in a sample as are, for instance, in vitro assays.

In a further embodiment, the methods and kit of the present invention may be designed or adapted to monitor immunotherapy reagents, immune function, disease states and effects of treatment and therapy. While the AR may be delivered to a target tissue residing in various regions of the test recipient’s body, optimal delivery sites will include those that are populated with endogenous mast cells that are located within the interstitial fluid of the target tissue. This provides an opportunity to detect cells expressing specific cell surface markers such as distinct immune cell populations. Detection or measurement of receptor binding ligands such as LAG3 cleavage, CD8/Treg ratios, cytokines, chemokines can be used to select and manage patients on immunotherapy. Chemotactic factors can be introduced concurrently or synchronously with the AR to attract cells of interest. As mentioned above, the delivery medium can include a chemotactic or other constitutes that draw cells of interest into the proximity of the AR once it has been delivered.

Further to the aforementioned, another embodiment of the invention provides a tool for exploring interstitial fluid by affording a methodology for collecting critical information present within interstitial and lymph fluid as well as providing for the development of specialized assays or treatments. It is almost impossible to collect significant amounts of interstitial fluid. The time-consuming nature of currently available procedures for removing and testing interstitial and lymph fluid introduces risks to patients and the need for medical expertise and specialized equipment limits the use of these procedures to basic research. Interstitial fluid and lymph contain unique biomarkers useful for detecting and diagnosing diseases and disease states, monitoring treatment and recovery, and for tracking patient well-being. The present invention can be adapted to ideally accomplish those objectives by fashioning the analyte binding moiety from an appropriate biomarker.

In a further embodiment, the bioassay of the present invention can be used for point-of-care and self-testing where the AR-bioassay is applied to a localized area of the skin (or other applicable areas) as a homogeneous assay, singularly or in multiplex, by needle prick or any intradermal injection method, including needleless systems. A blood sample is not required, and results can be read in a few minutes visually, by device or sensor.

In yet another embodiment, the present invention may be used for immediate measurement of troponin, providing a real-time measurement that would allow first-responders to provide patients suffering a potential myocardial infarction with immediate and life-saving care on-site or in transit. Such a troponin test can be designed for visual interpretation or for interpretation by a biosensor or other detection modality. A further aspect of this embodiment may include a transmitter possessing a delivery needle and/or sensor, separate or one in the same, that is applied to a patient. Test results corresponding to the presence or amount of troponin present in a subject are transmitted in real-time via the transmitter to a communication device such as a cell phone thereby providing a first responder or a remote medical staff with immediate results and the ability to monitor results should they change with time. Many tests considered STAT where immediate results could impact a patient’s well-being could be adapted to the present invention in such a manner. The use of sensors and other means for detecting the multivalent binding response is discussed in more detail below.

An additional embodiment of the invention pretains to a test for M. tuberculosis (Mtb), wherein the test directly detects Mtb activated T cells. An affinity reagent is constructed that contains at least one Fc.epsilon.Rl receptor binding domain and at least one MHC Class I molecule. The MHC molecule portion of the affinity reagent is paired with an Mtb antigen, forming a complex. The MHC molecule can be complexed with any suitable M. tuberculosis antigen. Preferably, the antigens are choosen from the secretory ESAT-6 protein, 10-kDa culture filtrate protein (CFP-10), or any of the proteins present in PPD preparations (Hodapp, T. et al., Eur Respir J 2012; 40: 152-160. doi: 10.1183/09031936.00175611; Urdahl, K.B. et al., Nature Review, VOL 4, No 3, MAY 2011). The affinity reagent is applied to a subject by any of the methods described herein. The affinity reagent binds to T cell receptors on T cells that have been activated by the presence of Mtb resulting in mast cell degranulation and a wheal and flare reaction that corresponds to the presence of Mtb.

Another embodimenet of the invention is a test to detect superantigens. Superantigens are implicated in T cell driven cytokine release syndrome (CRS) accompanied by multiple organ dysfunction (MOD). Superantigens bind directly to TCR and MHC-II receptors outside the conventional antigen-binding site and stimulate large number of T cells. Superantigens are implicated in Multisystem Inflammatory Syndrome in Children (MIS-C), COVID-19 cytokine storm syndrome, sepsis, toxic shock syndrome, chimeric antigen receptor T-cell (CART) therapy, administration of certain T cell agonistic antibodies and immune check point inhibitors.

An affinity reagent is constructed that contains at least one Fc.epsilon.Rl receptor binding domain and at least one MHC Class II molecule. A second affinity reagent is constructed that has at least one Fc.epsilon.Rl receptor binding domain and at least one T Cell Receptor (TCR) molecule. The paired affinity reagents are applied to the skin. The presence of superantigens will result in the cross-linking the MHC and TCR moieties of the affinity reagents leading to mast cell degranulation followed by a measurable wheal and flare reaction.

### Bioassay Kit

Minimally, the bioassay kit of the present invention includes the AR and a device for contacting mast cells in vivo with the AR. Use of the kit will be obvious to those skilled in the art upon reading the detailed description for the methods of the present invention. The kit may optionally include a sensor or other device for determining the presence, absence, or amount of an analyte of interest. The device utilized for contacting mast cells with the AR component of the kit can be any suitable device as described herein. The AR component of the kit may include any affinity reagents constructed in accordance with the methods set forth herein. The AR component will be pre-coated onto or pre-loaded into the delivery device as also described above. It is contemplated herein that in some cases, the AR may in be included in the kit in a lyophilized form for reconstitution with a suitable sterile carrier medium followed by subsequent application to the delivery end of the device just prior to initiating delivery of the AR to the target tissue of the subject. Optionally, the kit may include a device for detecting the multivalent binding response as described above.

### Sentinel Monitoring

The present invention also provides a method for the sentinel monitoring of an individual, wherein the affinity reagent is applied to the subject before the subject has been exposed to the analyte of interest or while the analyte is at undetectable concentrations. In this embodiment, even though the AR is delivered into the subject and reacts with mast cells in situ thereby sensitizing them to the analyte, crosslinking will not occur in the absence of the analyte. The mast cells that have become sensitized by the AR will stay sensitized for an extended period of time. Resident mast cells are long-lived cells that can survive for up to 12 weeks in the skin of Wistar rats or longer (Kiernan J.A, J Anat 128:225-238 (1979)). If the sensitized mast cells are subsequently exposed to the analyte of interest over the following days, weeks or months, the crosslinking that induces degranulation of the sensitized mast cells and other additional physiological changes to the target tissue resulting therefrom will then occur constituting the multivalent binding response which can be assessed to determine the presence or amount of the analyte of interest. For example, an AR molecule specific for the Ebola virus is delivered to a healthcare worker in the skin on the forearm before he or she enters the field to treat patients. If the healthcare worker is exposed at any time to Ebola, the Ebola virus or particles from the virus will bind to the sensitized mast cells eliciting a multivalent binding response which, in turn, may be assessed by evaluating the tissue at the delivery site where a wheal and flare reaction appears to alert the worker.

Modifications and variations of the present invention can be made without departing from its spirit and scope, as will be apparent to those skilled in the art. The specific examples described below are offered only to illustrate some embodiments of the present invention, such invention intended to be limited only by way of the appended claims, taken in conjunction with the full scope of equivalents to which such claims are entitled.

### EXAMPLES

### Example 1

### CD-1 Mice

Female CD-1 mice, retired breeders (-30 grams) were purchased from Charles River, 251 Ballardvale St., Wilmington, MA 01887. The mouse has played a historically important role in the dissection of the mechanisms of anaphylaxis in humans. Antigen-induced cross-linking of mast cell-associated IgE that leads to mast cell degranulation with release of mediators is highly conserved in mammals. Those knowledgeable in the art, would understand that the anaphylactic-based examples as encompassed in this invention and carried out in mice would translate to humans and other mammals (Ovary Z., Arerugi 43(12):1375 (1994)).

### Preparation of the HBsAg Affinity Reagent (HBsAg-AR)

The AR was constructed by recombinantly switching the Fc gamma region of an anti-HBsAg IgG antibody to that of an Fc epsilon region. Specifically, the HBsAg—AR was prepared from Anit-HBsAg 5C3 mouse IgG2a Fc silent antibody to contain the F1.epsilon.R1 receptor domain by switching the IgG isotype to IgE (mouse) and was carried out by Absolute Antibody Ltd, Wilton Centre, Redcar, Cleveland TS10 4RF, UK by proprietary methods. Anti-HBsAg 5C3 IgE was supplied in PBS with 0.2% Procline 300 and recognizes an antigenic determinant on HBsAg (Wands et al., Proc. Natl. Acad. Sci. USA 79:1277 (1982); Wands et al., Proc. Natl. Acad. Sci. USA 81:2237 (1984); Ben-Porath et al., J. Clin. Invest. 76:1338 (1985)).

### Hepatitis B Surface Antigen (HBsAg)

Recombinant full length HBsAg antigen (Subtype adw) produced in Saccharomyces cerevisiae (containing plasmid pCGA7) was purchased from Fitzgerald Industries International, 30 Sudbury Rd., Suite 1A North, Action, MA 01720. HBsAg was supplied in 0.05M phosphate, 0.2M NaCl, pH 7.2. Expression of HBsAg in yeast results in spherical and particles identical in size and shape to those particles (20-22 nm) found in HBV-infected patient’s sera (Miyanohara et al., Proc. Natl. Acad. Sci. USA Vol 80, pp. 1-5, January 1983). HBsAg itself has a molecular mass of ~24 kDa and the glycosylated form has a molecular mass of 27 kDa while the 22 nm particle has a molecular mass of 2.18 MDa (Ono et al., Nucleic Acids Research 11(6):1747 (1983); Miyanohara et al., Proc. Natl. Acad. Sci. USA 80:1 (1983).; Gilbert et al.," Proc. Natl. Acad. Sci. USA 102(41):14783 (2005)).

### Avertin Anesthetic

A stock solution was prepared from 99% 2,2,2-tribromoethanol (Acros Organics) and tert-amyl alcohol, reagent grade (Fisher Scientific). Ten ml tert-amyl alcohol was added to 10 g Avertin (2,2,2-tribromoethanol). The bottle in which the Avertin arrives is convenient for mixing. Drop in a stir bar and stir on a magnetic stirrer until Avertin is completely dissolved. This step took approximately 2 hours. The stock was kept in a dark bottle and tightly capped. It was stored at room temp. It should be noted that the Avertin stock is photosensitive and hydroscopic. Stock solution should be stable for 6-12 months. Working solution (1.2% Avertin) was prepared by adding 0.240 ml of stock to 19.76 ml of sterile water dropwise while water was being stirred by stir bar. Stirring was performed until the stock solution was completely dissolved. The stock solution was refrigerated and protected from light for use within 2-weeks. Avertin was administered IP using 0.800 ml per 30-gram mouse.

### Evans Blue

Due to its water solubility and slow excretion, as well as its tight binding to serum albumin, Evans Blue has been widely used in biomedicine to determine vascular permeability. When the release of histamine and other vasodilators from mast cells result in a disruption of the barrier and increased vascular permeability, Evans Blue-bounded albumin extravasates from the circulation into neighboring tissues. Leakage of dye across the blood vessel signifies a disintegration of the barrier, and the accumulation of Evans Blue dye can be quantified. A stock solution was prepared by adding 1 gram Evans Blue to 30 mls of water.

### Example 2

### Method for Detecting Analyte

Contact of the AR with analyte resulted in a visible and measurable localized inflammatory response that is directly proportional to the amount of analyte present in the subject. To demonstrate this, mice were injected intradermally with the HBsAg—AR to mast cells in vivo and then challenged intravenously with various amounts of HBsAg. Whereas, wheal and flare measurements alone can be performed in humans, mouse skin is very thin and measuring the leakage of the Evans blue around a positive wheal and flare reaction provided an easy method to visualize a positive reaction.

Mice under Avertin anesthesia were injected intradermally with 100 ng (10 ul) of HBsAg—AR in phosphate buffered saline (PBS) in the right ear pinna; mice received 10 ul of PBS intradermally in the left ear pinna as a control. The next day, mice were challenged intravenously with 100 ul of HBsAg in 1% Evans blue. Mice were challenged with 100 ug, 50 ug, 20 ug and 10 ug of HBsAg. HBsAg challenges were administered by retro-orbital injections. Reactions were measure at 20 minutes. Mice were categorized base on the surface area and density of Evans blue dye in the ear (Table 2).

Table 2

| | | | | |
|---|---|---|---|---|
| Challenge | 100ug | 50ug | 20ug | 10ug |
| Reaction | +++++ | ++++ | ++ | Neg |

### Example 3

### Enhanced Sensitivity

AR delivered to the target tissue was continually exposed to the analyte as the analyte circulated throughout the body until a measurable response occurred, thereby enhancing assay sensitivity. To demonstrate this, the mouse was challenged with 10 ug of HBsAg as described in Example 2 and was observed for the next 8 hours. The mouse registered as negative following 20 min to 4 hr exposure to the analyte but was clearly positive after 8 hours (Table 3).

Table 3

| | | | | | |
|---|---|---|---|---|---|
| Time | 20 min | 1 hour | 2 hours | 4 hours | 8 hours |
| Reaction | Neg | Neg | Neg | Neg | ++ |

### Example 4

### Ease of Use and Simplicity

The method of the present invention was performed in one step without the necessity to process samples by separation or washing steps, providing an easy to use homogeneous type format. To demonstrate this, mice with circulating HBsAg were tested per the method of the present invention wherein the AR was delivered both intradermally and by needle prick. Mice under Avertin anesthesia were challenged with 100 ug HBsAg diluted in 100 ul of 1% Evans Blue as described above. HBsAg challenges were administered by retro-orbital injections. Injected HBsAg was allowed to pre-circulate for 10 min prior to the intradermally application of the HBsAg—AR in the right ear pinna as described above. HBsAg—AR was also pipetted onto the surface of the left ear and a needle was used to pierce the surface layer of the skin. Reaction times were observed and recorded in Table 4. Intradermal application of the AR resulted in a dense blue patch covering 30% to 40% of the ear. Needle prink resulted in small discrete and dense blue patches.

Table 4

| | | | | |
|---|---|---|---|---|
| Time | 30 sec | 2 min | 3 min | 5 min |
| Intradermal | +/- | +++ | ++++ | ++++ |
| Needle Prick | +/- | +++ | ++++ | ++++ |

### Example 5

### Sentinel Test

To demonstrate this, Mice under Avertin anesthesia were injected intradermally with 100 ng (10 ul) of HBsAg—AR in PBS in the right ear pinna; mice received 10 ul of PBS intradermally in the left ear pinna as a control as described above. Seven days later, Mice under Avertin anesthesia were challenged with 100 ug HBsAg diluted in 100 ul of 1% Evans Blue as described above. After 16 days, anesthetized mice were challenged with 30 ug HBsAg diluted in 50 ul of 1% Evans Blue as described above. HBsAg challenges were administered by retro-orbital injections. Reactions were measured at 20 minutes. The results demonstrated that the method could be used to detect exposure to analytes 7 and 10-days after the AR mast cells, in situ, are exposed to AR (Table 5).

Table 5

| | | |
|---|---|---|
| Time | 7-Days | 16-Days |
| Reaction | ++++ | ++++ |

### Example 6

### Efficacy in Humans and Sentinel Test

A recombinantly produced affinity reagent that detected Ara h 2 was made by Absolute Antibody using proprietary methods. In this case, the affinity reagent was essentially an IgE antibody with specificity to an epitope on Ara h 2 allergen. The reagent possessed the required Fc.epsilon.Rl binding domain and the additional moiety that detected Ara h 2. The affinity reagent (1 mg/ml) was supplied in PBS, 0.2% Procline 300.

An intradermal injection was performed on the forearm of the applicant with 100 ng (10 ul) of Ara h 2-affinity reagent in PBS (protocol #1). Additionally, the affinity reagent was applied using a Unitest PC skin test applicator (Lincoln Diagnostics, Inc., Decatur, IL 62526 ) using 2 different procedures. In the first procedure, ~ 20 ul of affinity reagent was pipetted onto the skin in PBS, and the Unitest PC applicator administered through the affinity reagent into the dermal layer of the skin (protocol #2). In the second procedure, the affinity reagent was suspended in 50% glycerol in PBS allowing the affinity reagent to be absorbed onto the tip of the Unitest PC by capillary action. The Unitest PC applicator was then applied to the dermal layer of the skin in one step (protocol #3). A 50 ug/ml concentration of affinity reagent was used with the Unitest device.

Peanuts were consumed at 30 min intervals for 6 hours. The assay using protocol #1, #2, and #3 was applied to the forearm and the reaction measured after 20 min. Controls were included for each protocol consisting of the application vehicle without the affinity reagent. A distinct wheal and flare was clearly visible using all three protocols where the affinity reagent was applied, while negative control showed no sign reaction (Table 6).

Ara 2 h was eliminated from the applicant’s blood by waiting 24 hr after the last peanuts were consumed. The assay was then applied using protocol #2 and the reaction measured after 20 min. To demonstrate feasibility of a sentinel test, the assay was applied to the applicant prior to consuming peanuts. The assay was negative and remained negative for 24 hours. Because of the lag period between eating peanuts and the appearance of Ara 2 h in the blood, test results were measured 20 minutes after the first sign of a reaction (Table 7).

Table 6

| | Protocol #1 (mm) | Protocol #2 (mm) | Protocol #3 (mm) |
|---|---|---|---|
| Control | 0.0 | 0.0 | 0.0 |
| +Ara h 2 | 12.00 | 8.5 | 7.0 |

Table 7

| | Protocol #2 (mm) |
|---|---|
| -Ara h 2 | 0.0 |
| +Ara h 2 (after 24 hrs) | 7.0 |

### Example 7

### Human Sentinel Test

The affinity reagent (anti-Ara h 2, IgE) was prepared as described above and applied in ~5 to 10 ul of saline to the forearm of the applicant by intradermal injection creating a bubblelike area. The bubblelike area disappeared in a few hours and all traces of the injection site were gone by the next day. Peanuts were consumed by the applicant 1 week later. Within 30 min of consumption a tingle at the site was present, followed by a slight sting. With a few minutes a distinct wheal and flare reaction appeared and remained for approximately 2 hours. The wheal measured 20 mm within 20 min of the first signs of a reaction (Table 8).

Table 8

| | |
|---|---|
| | Reaction |
| -Ara h 2 | 0.0 mm |
| +Ara h 2 (after 1 wk) | 20.0 mm |

### Example 8

### Application of Affinity Reagent Dried onto Applicator

The affinity reagent (anti-Ara h2, IgE) was dried onto the tip of a Unitest PC applicator. A 50 ul aliquot of a 1:10 dilution of stock affinity reagent into PBS was placed in a 1.5 ml microfuge tube. The Unitest PC applicator was placed in the microfuge tube with the tip submerged in the affinity reagent for 1 or 2 minutes. The applicator was removed and allowed to dry for 4 hours. The applicator was applied to the subject’s arm for 30 seconds. There was no blood visible, but the imprint of the applicator was present showing that the applicator had been properly applied to the forearm as indicated by the manufacturers user’s guide. All marks of the applied test had disappeared within 30 min. The subject consumed peanuts 24 hours later. Within 20 min a slight tingling occurred followed by a low-grade stinging sensation at which time a wheal and flare reaction was observed. The wheal and flare was at maximum intensity at 40 min post-consumption. A distinct wheal and flare reaction of 8 mm was present and persisted for over 2 hours (Table 9). After 2 hours the wheal and flare diminished and only a remnant of the reaction remained over the next 6 hours.

Table 9

| | |
|---|---|
| | Reaction |
| -Ara h 2 | 0.0 mm |
| +Ara h 2 (applied dry) | 8.0 mm |

While the foregoing disclosure taken together with the references incorporated herein relates to and describes certain embodiments that are provided to enable and exemplify the concept of the invention as well as certain exemplary instruction as to reduction to practice, those skilled in the art will appreciate that changes and modifications to the foregoing may be made without departing from the spirit of the invention and without undue experimentation. The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Accordingly, it is intended to claim all such changes and modifications as fall within the true scope of the invention.

We claim:

1. A method for determining a presence, absence, or amount of an analyte in a living mammal comprising method steps of:
providing at least one affinity reagent construct comprising at least one Fc.epsilon.R1 receptor binding domain and at least one additional moiety capable of binding said analyte wherein the resulting construct binds both a mast cell and said analyte in any order; contacting at least one endogenous mast cell in situ and present within a target tissue of said mammal with said reagent construct to elicit a multivalent binding response when said analyte is present; and
performing an assessment of said multivalent binding response to determine said presence or amount of said analyte.

2. The method of claim 1 wherein said contacting further comprises delivering said affinity reagent construct into a skin of said mammal.

3. The method of claim 1 wherein said contacting further comprises delivering said affinity reagent construct into tissue of said mammal wherein said affinity reagent is exposed to from about 125 to 20,000 mast cells per cubic millimeter.

4. The method of claim 3 wherein said delivering further comprising performing delivery with at least one device selected from the group consisting essentially of a needle prick, intradermal injection, solid needle, hollow needle, patch method or needleless system.

5. The method of claim 2 wherein said contacting step further comprises delivering said affinity reagent construct into the dermal layer.

6. The method of claim 1 wherein performing said assessment further comprises evaluating said target tissue for at least one physiological change in said target tissue.

7. The method of claim 6 further comprising evaluating a morphological change in said target tissue to render assessment of said multivalent binding response.

8. The method of claim 6 further comprising using a device to measure a morphological change.

9. The method of claim 6 further comprises using a sensor for evaluation of said target tissue to render assessment of said multivalent binding response.

10. The method of claim 7 further comprising performing a visual inspection for a wheal and flare reaction.

11. The method of claim 8 wherein said device measures a wheal and flare reaction.

12. The method of claim 1 wherein said constructing step further comprises using at least one technique selected from the group consisting essentially of hybridoma technology, covalent conjugation, non-covalent binding and genetic engineering methods.

13. The method of claim 1 wherein said constructing step further comprises fashioning said additional moiety using at least one member from the group consisting essentially of antibodies, recombinant antibodies, engineered antibodies, antibody fragments, synthetic antibodies, engineered non-antibody binding proteins, antigens, chimeric molecules, fusion proteins, aptamers, hormones, receptors, receptor binding molecules, drugs, toxicants, toxins, pathogens, pathogen components, biomarkers, cell surface markers, ligands, RNA, or DNA.

14. The method of claim 1 wherein performing an assessment further comprises measuring a substance released during mast cell degranulation.

15. The method of claim 1 wherein said analyte is Mycobacterium tuberculosis and said additional moiety comprises at least one major histocompatibility complex protein complexed with at least one Mycobacterium tuberculosis antigen.

16. The method of claim 15 wherein said major histocompatibility complex protein is selected from the group consisting of MHC Class I and MCH Class II proteins.

17. The method of claim 15 wherein said Mycobacterium tuberculosis antigen is either Mtb secretory antigenic target ESAT-6 or 10-kDa culture filtrate protein CFP-10.

18. The method of claim 1 wherein said analyte is selected from SARS-CoV-2 antibodies and said additional moiety comprises an antigen derived from SARS-CoV-2 virus.

19. The method of claim 18 wherein said antigen is derived from a protein selected from the group consisting of SARS-CoV-2 receptor binding domain, spike protein, spike S 1 protein, spike S2 protein and SARS-CoV-2 nucleocapsid protein.

20. A method of testing for a suspected pathology in a living mammal comprising method steps of:
selecting an analyte indicative of said suspected pathology;
providing at least one affinity reagent construct comprising at least one Fc.epsilon.R1 receptor binding domain and at least one additional moiety capable of binding said analyte wherein the resulting construct binds both a mast cell and said analyte in any order;
contacting at least one endogenous mast cell in situ and present within a target tissue of said mammal with said reagent construct to elicit a multivalent binding response when said analyte is present; and
performing an assessment of said multivalent binding response to determine said presence or amount of said analyte.

21. A method of testing a living mammal for exposure to at least one substance selected from the group consisting essentially of chemicals, drugs, biowarfare agents, toxicants, toxins and pathogens, the method comprising method steps of:
selecting an analyte indicative of exposure to said substance;
providing at least one affinity reagent construct comprising at least one Fc.epsilon.R1 receptor binding domain and at least one additional moiety capable of binding said analyte wherein the resulting construct binds both a mast cell and said analyte in any order;
contacting at least one endogenous mast cell in situ and present within a target tissue of said mammal with said reagent construct to elicit a multivalent binding response when said analyte is present; and
performing an assessment of said multivalent binding response to determine said presence or amount of said analyte.

22. An affinity reagent for an in vivo bioassay, said reagent comprising at least one Fc.epsilon.R1 receptor binding domain and at least one additional moiety capable of bindingan analyte of interest, said reagent capable of binding both a mast cell and said analyte in any order.

23. The affinity reagent of claim 22 wherein the additional moiety is selected from the group consisting essentially of antibodies, recombinant antibodies, engineered antibodies, antibody fragments, synthetic antibodies, engineered non-antibody binding proteins, antigens, chimeric molecules, fusion proteins, aptamers, hormones, receptors, receptor binding molecules, drugs, toxicants, toxins, pathogens, pathogen components, biomarkers, cell surface markers, ligands, RNA and DNA.
